(19) United States
(12) Patent Application Publication
(10) Pub. No.: US 2018/0161366 A1
(43) Pub. Date: Jun. 14, 2018

(54) METHODS OF OBTAINING MONONUCLEAR BLOOD CELLS AND USES THEREOF

(71) Applicants: Biokine Therapeutics Ltd., Nes Ziona (IL); BioLineRx Ltd., ModiIn (IL)

(72) Inventors: Amnon PELED, Tel-Aviv (IL); Yaron PEREG, Shoham (IL)

(73) Assignees: Biokine Therapeutics Ltd., Nes Ziona (IL); BioLineRx Ltd., ModiIn (IL)

(21) Appl. No.: 15/571,069

(22) PCT Filed: May 19, 2016
(86) PCT No.: PCT/IL2016/050529

§ 371 Date: Nov. 1, 2017

Related U.S. Application Data
(60) Provisional application No. 62/164,076, filed on May 20, 2015.
(60) Provisional application No. 62/164,024, filed on May 20, 2015.

(51) Int. Cl. A61K 35/15; C12N 5/0784; C12N 5/0783; C12N 5/0781; C12N 5/0775; A61K 35/17; A61K 45/06; A61P 35/00; A61K 38/10; A61K 35/14; A61K 35/28

(52) U.S. Cl. CPC A61K 35/15; C12N 5/0639; C12N 5/0636; C12N 5/0635; C12N 5/0665; A61K 35/17; C12N 2501/24; A61P 35/00; A61K 38/10; A61K 35/14; A61K 35/28; C12N 2501/2302; C12N 2501/2312; A61K 45/06

(57) ABSTRACT

Methods of obtaining mononuclear blood cells are provided. Also provided are methods of using the obtained cells for treating diseases such as cancer, infectious disease, autoimmune disease, allergy, and graft rejection.

### FIELD AND BACKGROUND OF THE INVENTION

The present invention, in some embodiments thereof, relates to methods of obtaining mononuclear blood cells and, more particularly, but not exclusively, to their use in the treatment of cancer.

Cancer is the second leading cause of death in the US. The estimates for 2014 are that approximately 585,000 people will die of cancer and 1.6 million new cases will be diagnosed (American Cancer Society, Cancer Facts & FIGS. 2014). For early stage cancers, surgical removal is a very effective treatment. However, for more advanced cases and non-solid hematological malignancies, standard, non-specific cancer treatments such as chemotherapy and radiotherapy are typically used. These treatments affect many healthy cells and result in elevated toxicity and effective in only a minor percentage of treated individuals. Moreover, even individuals that initially respond to therapy are at risk for relapses, and often develop resistance.

Significant progress in understanding the underlying principles of tumor biology as well as the basic mechanisms of the immune response to cancer have led to the development of new immunotherapies aimed at employing the adaptive immune system to eradicate cancer with enhanced efficacy and reduced toxicity. Current immunotherapy strategies include cytokines, monoclonal antibodies against tumor cells or immune regulatory molecules, cancer vaccines and cell-based therapies such as adoptive transfer of ex-vivo activated T cells and natural killer (NK) cells.

4F-benzoyl-TN14003 (also known as BKT140, hereinafter BL-8040), is a 14-residue bio stable synthetic peptide developed as a specific CXCR4 antagonist. It has been shown that BL-8040 binds the CXCR4 receptor with high affinity and long receptor occupancy. Studies in mice demonstrated that a single BL-8040 injection mobilized long term repopulating stem cells sufficient for transplantation. [Abraham M et al., Stem Cells (2007); 25:2158-66]. Results from a study in multiple myeloma patients showed that combined treatment of BL-8040 and G-CSF enabled the collection of high number of CD34+ hematopoietic stem/progenitor cells (HSPC) in a single aphaeresis procedure.[Peled A et al. Clin Cancer Res; (2013) 20(2); 469-79]. In addition, BL-8040 was found to be toxic against several tumors such as myeloid leukemia, hematopoietic tumors and non-small cell lung cancer (International Patent Application No. IL2014/050939 and International Patent Application Publication No. WO2013/16089556053 and WO2008/075370).

Additional Background Art Includes:

International Patent Application Publication No. WO2014/155376;

International Patent Application Publication No. WO2012/095849;

International Patent Application Publication No. WO2002/20561;

International Patent Application Publication No. WO2004/020462;

International Patent Application Publication No. WO2008/075369;

International Patent Application Publication No. WO2008/075371;

International Patent Application Publication No. WO2010/146578;

International Patent Application Publication No. WO2010/146584;

International Patent Application Publication No. WO2003/072599;

International Patent Application Publication no. WO 2015/019284; and

U.S. Patent Application Publication No. US 2012/0082687.

### SUMMARY OF THE INVENTION

According to an aspect of some embodiments of the present invention there is provided a method of obtaining dendritic cells (DCs) from a subject, the method comprising:

(a) administering to the subject an effective amount of a peptide having an amino acid sequence as set forth in SEQ ID NO:1 or an analog or derivative thereof; and

(b) collecting peripheral blood of the subject 4-8 hours following the administering;

thereby obtaining the DCs from the subject.

According to an aspect of some embodiments of the present invention there is provided a method of obtaining at least one type of mononuclear blood cells (MNBCs) selected from the group consisting of T cells, B cells, NK cells and NKT cells from a subject, the method comprising:

(a) administering to the subject an effective amount of a peptide having an amino acid sequence as set forth in SEQ ID NO: 1 or an analog or derivative thereof;

(b) collecting peripheral blood of the subject 4-48 hours following the administering; and optionally;

(c) repeating step (b) at least once no later than 48 hours following the administering;

thereby obtaining the at least one type of MNBCs from the subject.

According to some embodiments of the invention, the MNBCs comprise T cells.

According to some embodiments of the invention, the method further comprising purifying the MNBCs from the peripheral blood following the collecting.

According to an aspect of some embodiments of the present invention there is provided a method of obtaining cells effective for the treatment of cancer, the method comprising:

(a) administering to a subject an effective amount of a peptide having an amino acid sequence as set forth in SEQ ID NO: 1 or an analog or derivative thereof;

(b) collecting peripheral blood of the subject; and

(c) enriching from the peripheral blood at least one type of mononuclear blood cells (MNBCs) capable of eliciting an immune response against a cancerous cell,

thereby obtaining the cells effective for the treatment of cancer.

According to some embodiments of the invention, the enriching comprises purifying at least one type of MNBCs selected from the group consisting of dendritic cells (DCs), T cells, B cells, NK cells and NKT cells from the peripheral blood following the collecting.

According to some embodiments of the invention, the cells are selected from the group consisting of dendritic cells (DCs), T cells, B cells, NK cells and NKT cells.

According to some embodiments of the invention, the cells comprise dendritic cells (DCs).

According to some embodiments of the invention, the DCs comprise immature DCs.

According to some embodiments of the invention, when the cells comprise immature DCs the method comprises inducing maturation of the immature DCs.

According to some embodiments of the invention, the cells comprise T cells.

According to some embodiments of the invention, the enriching is effected by a method selected from the group consisting of:

(i) selecting anti-cancer reactive cells;

(ii) activating anti-cancer reactive cells;

(iii) expanding anti-cancer reactive cells;

(iv) promoting presentation of a cancer antigen; and

(v) promoting presentation of an anti-cancer receptor.

According to some embodiments of the invention, the enriching comprises contacting the peripheral blood or a purified population of cells thereof with a cancer antigen selected from the group consisting of a cancer antigenic peptide or polypeptide, a cancer cell lysate, a cancerous cell and a DC presenting a cancer antigen.

According to some embodiments of the invention, the activating or expanding comprises contacting the peripheral blood or a purified population of cells thereof with a cytokine capable of inducing activation and/or proliferation of a T cell.

According to some embodiments of the invention, the activating comprises contacting the peripheral blood or a purified population of cells thereof with a co-stimulatory molecule.

According to some embodiments of the invention, the co-stimulatory molecule is selected form the group consisting of an immune-check point regulator, LPS and TLR ligands.

According to some embodiments of the invention, the immune-check point regulator is selected from the group consisting of anti-CTLA4, anti-PD-1 and CD40 agonist.

According to some embodiments of the invention, when the cells comprise T cells, the promoting presentation of an anti-cancer receptor comprises transducing with a T cell receptor (TCR) or a chimeric antigen receptor (CAR).

According to some embodiments of the invention, when the cells comprise DCs the promoting presentation of a cancer antigen comprises transfecting with an mRNA coding for a cancer antigen.

According to some embodiments of the invention, there is provided a method of treating cancer in a subject in need thereof, the method comprising:

(a) obtaining cells effective for the treatment of cancer according to the method; and

(b) transplanting the cells to a subject,

thereby treating the cancer in the subject.

According to some embodiments of the invention, when the cells comprise DCs, the transplanting is in combination with an adjuvant.

According to some embodiments of the invention, the transplanting is in combination with an anti-cancer immune modulator agent.

According to some embodiments of the invention, the transplanting is effected prior to the treatment with the agent.

According to some embodiments of the invention, the transplanting is effected concomitant with the treatment with the agent.

According to some embodiments of the invention, the transplanting is effected following the treatment with the agent.

According to some embodiments of the invention, the agent is selected from the group consisting of a cancer antigen, a cancer vaccine, an anti-cancer antibody, a cytokine capable of inducing activation and/or proliferation of a T cell and an immune-check point regulator.

According to some embodiments of the invention, the cytokine is selected from the group consisting of IL-2, IFNα and IL-12.

According to some embodiments of the invention, the immune-check point regulator is selected form the group consisting of anti-CTLA4, anti-PD-1, anti-PDL-1, CD40 agonist, 4-1BB agonist, GITR agonist and OX40 agonist.

According to an aspect of some embodiments of the present invention there is provided a method of obtaining cells effective for the treatment of an infectious disease, the method comprising:

(a) administering to a subject an effective amount of a peptide having an amino acid sequence as set forth in SEQ ID NO:1 or an analog or derivative thereof;

(b) collecting peripheral blood of the subject; and

(c) obtaining from the peripheral blood at least one type of mononuclear blood cells (MNBCs) capable of eliciting an immune response against a pathogen, thereby obtaining the cells effective for the treatment of the infectious disease.

According to some embodiments of the invention, the cells are selected from the group consisting of memory T cells, pathogen-specific T cells and DCs presenting a pathogenic antigen.

According to some embodiments of the invention, there is provided a method of treating an infectious disease in a subject in need thereof, the method comprising:

(a) obtaining cells effective for the treatment of an infectious disease in a subject according to the method; and

(b) transplanting the cells to a subject,

thereby treating the infectious disease in the subject.

According to an aspect of some embodiments of the present invention there is provided a method of obtaining cells effective for treatment of an autoimmune disease, allergy or graft rejection disease, the method comprising:

(a) administering to a subject an effective amount of a peptide having an amino acid sequence as set forth in SEQ ID NO:1 or an analog or derivative thereof;

(b) collecting peripheral blood from the subject;

(c) obtaining from the peripheral blood at least one type of mononuclear blood cells (MNBCs) capable of inducing tolerance to an autoimmune cell, an allergen or a graft;

thereby obtaining the cells effective for treatment of the autoimmune disease, allergy or graft rejection disease.

According to some embodiments of the invention, the cells are selected from the group consisting of regulatory DCs, immature DCs and regulatory T cells.

According to some embodiments of the invention, there is provided a method of treating an autoimmune disease, allergy or graft rejection disease in a subject in need thereof, the method comprising:

(a) obtaining cells effective for treatment of an autoimmune disease, allergy or graft rejection disease in a subject according to the method; and

(b) transplanting the cells to a subject,

thereby treating the autoimmune disease, allergy or graft rejection disease in the subject.

According to some embodiments of the invention, there is provided a method of transplanting a graft in a subject in need, the method comprising:

(a) transplanting the graft in the subject;

(b) obtaining cells according to the method; and

(c) transplanting the cells to the subject,

thereby transplanting the graft in the subject.

According to some embodiments of the invention, the graft is a hematopoietic stem cell graft.

According to some embodiments of the invention, the graft and the cells are syngeneic.

According to some embodiments of the invention, step (a) is effected prior to step (c).

According to some embodiments of the invention, step (a) is effected following step (c).

According to some embodiments of the invention, step (a) is effected concomitantly with step (c).

According to some embodiments of the invention, the cells are selected from the group consisting of T cells transduced with a suicide gene, pathogen-specific T cells, leukemia or lymphoma-specific T cells, non-alloreactive T cells, veto cells, TCRγδ⁺ T cells and regulatory T cells.

According to some embodiments of the invention, the transplanting is autologous transplantation.

According to some embodiments of the invention, the transplanting is non-autologous transplantation.

According to some embodiments of the invention, the collecting is effected by leukapheresis. According to some embodiments of the invention, the method further comprising purifying the at least one type of MNBCs following the enriching.

According to some embodiments of the invention, the purifying is effected by a method selected from the group consisting of FACS and magnetic bead separation.

According to some embodiments of the invention, the collecting is effected 4-48 hours following the administering.

According to some embodiments of the invention, the collecting is effected 4-8 hours following the administering.

According to some embodiments of the invention, the peptide is administered at a dose of 0.5-1 mg/kg.

According to some embodiments of the invention, the peptide is administered subcutaneously.

According to some embodiments of the invention, the peptide is as set forth in SEQ ID NO: 1.

According to some embodiments of the invention, the cells do not comprise CD34+ hematopoietic stem/progenitor cells.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a graph illustrating kinetic analysis of the effect of BL-8040 administration on mobilization of white blood cells (WBC) to the peripheral blood. The figure presents a time course of total WBC in the peripheral blood of subjects in response to a single SC administration of 1 mg/kg BL-8040 as determined by flow cytometry.

FIG. 2 is a graph illustrating kinetic analysis of the effect of BL-8040 administration on mobilization of immature dendritic cells (ImDC) to the peripheral blood. The figure presents a time course of ImDC in the peripheral blood of subjects in response to a single SC administration of 1 mg/kg BL-8040 as determined by flow cytometry.

FIG. 3 is a graph illustrating kinetic analysis of the effect of BL-8040 administration on mobilization of CD3+ T cells to the peripheral blood. The figure presents a time course of CD3+ T in the peripheral blood of subjects in response to a single SC administration of 1 mg/kg BL-8040 as determined by flow cytometry.

FIG. 4 are graphs presenting the number of CD3+ T cells and B220+ B cells in spleen and peripheral lymph nodes (PLN) of C57BL mice prior (denoted as control) and following administration of 5 mg/kg BL-8040, as determined by flow cytometry.

FIG. 5 is a schematic illustration showing some embodiments in the development of a novel BL8040-based adoptive transfer therapy.

According to a specific embodiment, BL8040 is injected at 0.25-3.0 mg/Kg and cells are collected from the blood 2-24 hr later. The mononuclear cells are purified e.g., on Ficoll and incubated on irradiated tumor cells or tumor antigens in the presence of cytokines such as IL-2 and IL-15 and GM-CSF and antibodies to immune checkpoints such as anti PD-1 for 1-30 days. Expended cells are collected and administrated into patients with or without cytokines or other immunomodulators such as anti PD-1

FIGS. 6A-B show differential BL8040-induced mobilization of T cells subsets in naïve mice or pancreatic and or cancerous donors. Naïve mice or mice bearing pancreatic or melanoma cancer were injected with BL8040 (400 μg/mouse S.C.). FIG. 6A—shows increased mobilization of both CD4 and CD8 cells in tumor bearing mice compare to control non injected mice or control naïve nice which were either injected with BL8040 or not. FIG. 6B-shows that cells collected from cancerous mice contained more CD69+ CD25+ activated T cells.

FIGS. 7A-C Equal number of PBMC cells that were collected following mobilization with BL-8040 or from non mobilized mice were seeded on irradiated pancreatic cancer cells in the presence of IL-2 and anti PD1 for 11 days. FIG. 7A-shows that in all cultures CD8+ cells proliferated selectively in the cultures. FIG. 7B-shows that cells collected from naïve mice and mice with pancreatic cancer were activated CD69+ CD25+ cells. Cells collected from mice with pancreatic tumors showed a prominent LAGS expression (marker for TILLS). FIG. 7C-shows that cells collected from pancreatic tumors were mainly effector T cells (CD44+) whereas cells collected from naïve mice were mainly intermediate T cells (CD44−).

### DESCRIPTION OF SPECIFIC EMBODIMENTS OF THE INVENTION

The present invention, in some embodiments thereof, relates to methods of obtaining mononuclear blood cells and, more particularly, but not exclusively, to their use in the treatment of cancer.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Treatment for most types of cancer involves cytotoxic treatments such as chemotherapy and radiotherapy that may at least partially affect many healthy cells and thus result in elevated toxicity. In addition, these treatments are effective in only a small percentage of cancer affected patients. Immunotherapy strategies for cancer therapy, aiming at enhancing efficacy and reducing toxicity, include cytokines, monoclonal antibodies against tumor cells or immune regulatory molecules, cancer vaccines as well as cell-based therapies such as adoptive transfer of ex-vivo activated T cells and natural killer (NK) cells.

4F-benzoyl-TN14003 (SEQ ID NO: 1, also known as BKT140, hereinafter BL-8040) is a CXCR4 antagonist belonging to the T-140 peptide family. It has been shown that BL-8040 induces mobilization of CD34+ hematopoietic stem/progenitor cells (HSPC) that can be further used for transplantation. In addition, BL-8040 was found to be toxic against several tumors such as myeloid leukemia, hematopoietic tumors and non-small cell lung cancer.

While reducing the present invention to practice, the present inventors have surprisingly uncovered that in-vivo administration of BL-8040 induces a unique pattern of mononuclear blood cells (MNBCs) mobilization to the peripheral blood.

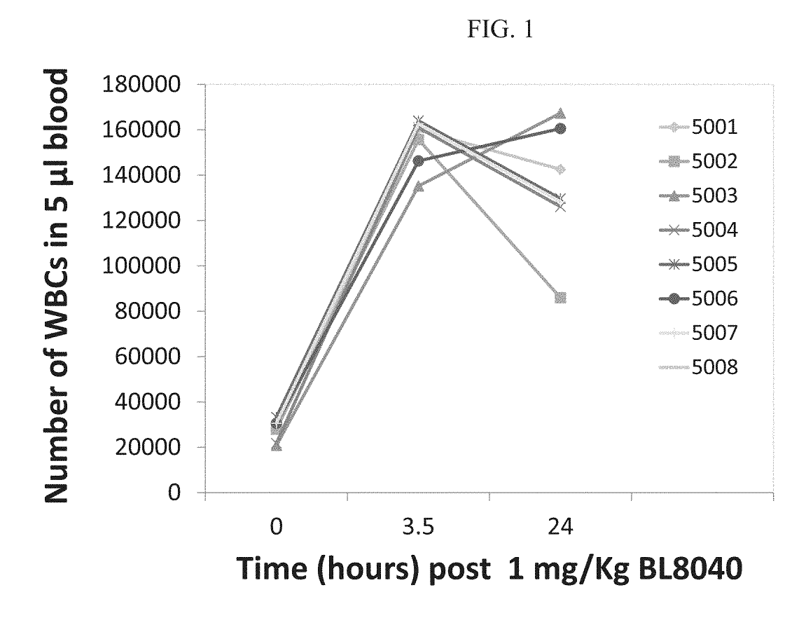

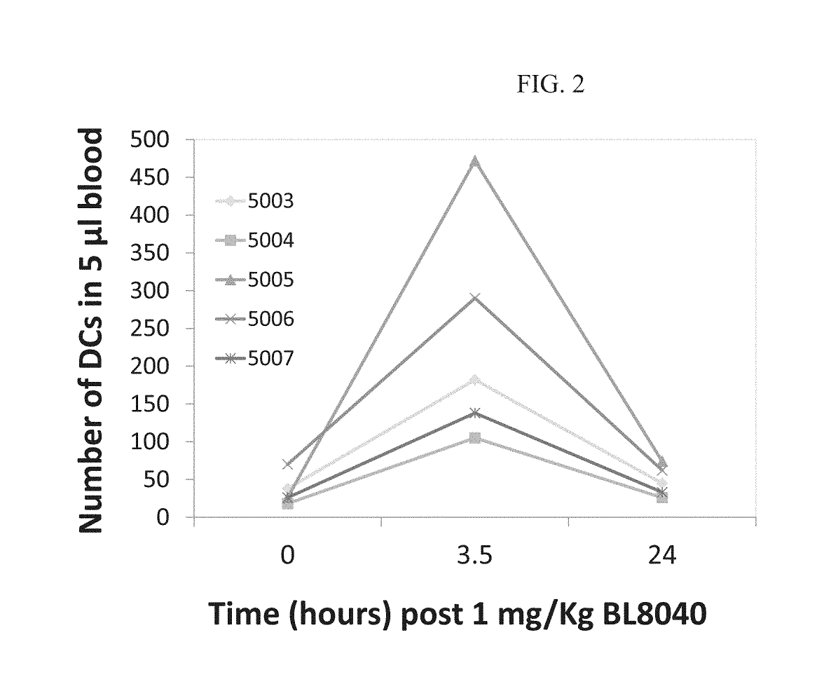

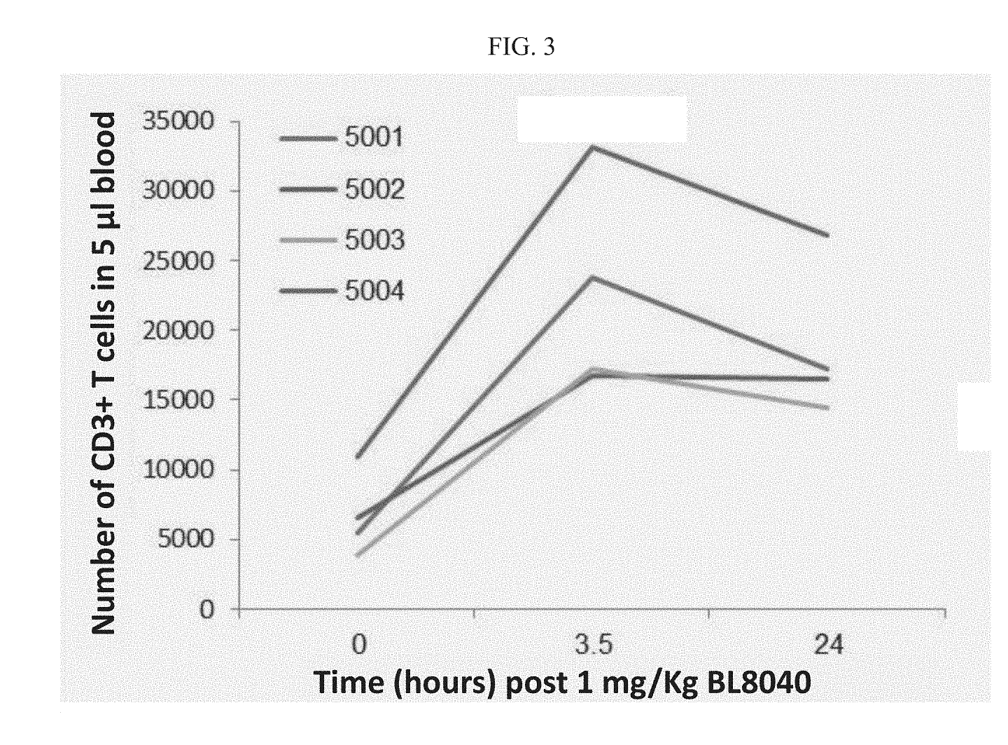

As is illustrated hereinbelow and in the examples section, which follows, the present inventors show that administration of BL-8040 triggered mobilization of total white blood cells to the peripheral blood of the treated subject (Example 1, FIG. 1). The present inventors have further uncovered that a single administration of BL-8040 is capable of stimulating the mobilization of several subtypes of MNBCs, including CD3+ T cells and immature dendritic cells (Example 1, FIGS. 2-3). The kinetics of mobilization was surprisingly found to be cell-specific. For instance, the mobilization of DCs and T cells is rapid and occurs within about 3-4 hours; interestingly while the number of DCs in the periphery decline rapidly and reach the basal level within 24 hours following BL-8040 administration, the number of T cells in the periphery remains high even 24 hours following administration of BL-8040.

Whilst further reducing the present invention to practice, the present inventors have devised a novel protocol for obtaining cells effective for the treatment of cancer. The protocol is based on administering to a subject an effective amount of a BL-8040 peptide or an analog or derivative thereof; collecting peripheral blood of the subject; and ex-vivo enriching from the peripheral blood at least one type of mononuclear blood cells (MNBCs) capable of eliciting an immune response against a cancerous cell, wherein enriching is done by subjecting the cells to tumor antigens in the presence of cytokines and optionally immune check point regulators (e.g., anti-PD1).

Consequently, the present teachings and the protocols presented in Examples 4, suggest the use of BL-8040 to obtain MNBCs or specific subsets thereof that can be used for adoptive transfer therapies or for the development of vaccines for the treatment of several diseases such as cancer, infectious disease, autoimmune disease and graft rejection.

Thus, according to an aspect of the present invention there is provided a method of obtaining at least one type of mononuclear blood cells (MNBCs) selected from the group consisting of T cells, B cells, NK cells and NKT cells from a subject, the method comprising:

(a) administering to the subject an effective amount of a peptide having an amino acid sequence as set forth in SEQ ID NO:1 or an analog or derivative thereof;

(b) collecting peripheral blood of said subject 4-48 hours following said administering; and optionally

(c) repeating step (b) at least once no later than 48 hours following said administering;

thereby obtaining the at least one type of MNBCs from the subject.

According to another aspect of the present invention there is provided a method of obtaining dendritic cells (DCs) from a subject, the method comprising:

(a) administering to the subject an effective amount of a peptide having an amino acid sequence as set forth in SEQ ID NO:1 or an analog or derivative thereof; and

(b) collecting peripheral blood of said subject 4-8 hours following said administering;

thereby obtaining the DCs from the subject.

As used herein the term “mononuclear blood cells (MNBCs)” refers to a blood cell having a single nucleus and includes lymphocytes, monocytes and dendritic cells (DCs).

According to specific embodiments the MNBCs are selected from the group consisting of dendritic cells (DCs), T cells, B cells, NK cells and NKT cells. According to specific embodiments the cells comprise DCs.

As used herein the term “DCs” refers to an antigen presenting cells capable of sensitizing HLA-restricted T cells. DCs include DCs derived from bone marrow hematopoietic cells such as plasmacytoid dendritic cells, myeloid dendritic cells, Langerhans cells and interdigitating cells; and follicular DCs. Dendritic cells may be recognized by function, or by phenotype, particularly by cell surface phenotype. These cells are characterized by their distinctive morphology having veil-like projections on the cell surface, intermediate to high levels of surface HLA-class II expression and ability to present antigen to T cells, particularly to naïve T cells (See Steinman R, et al., Ann. Rev. Immunol. 1991; 9:271-196.). Typically, cell surface phenotype of DCs include CD1a+, CD4+, CD86+, or HLA-DR. The term DCs encompasses both immature and mature DCs.

According to a specific embodiment the DCs comprise immature DCs. Specific cell surface phenotype of immature DCs may be lin-/CD11c+/CD83−.

According to other specific embodiments the MNBCs comprise T cells, B cells, NK cells and NKT cells.

According to specific embodiments the cells obtained according to the methods of the present invention comprise T cells.

As used herein, the term “T cells” refers to a differentiated lymphocyte with a CD3⁺, T cell receptor (TCR)⁺ having either CD4⁺ or CD8⁺ phenotype. The T cell may be either an effector or a regulatory T cells.

As used herein, the term “effector T cells” refers to a T cell that activates or directs other immune cells e.g. by producing cytokines or has a cytotoxic activity e.g., CD4+, Th1/Th2, CD8+ cytotoxic T lymphocyte.

As used herein, the term “regulatory T cell” or “Treg” refers to a T cell that negatively regulates the activation of other T cells, including effector T cells, as well as innate immune system cells. Treg cells are characterized by sustained suppression of effector T cell responses. According to a specific embodiment, the Treg is a CD4+CD25+Foxp3+ T cell.

According to specific embodiments, the T cells are CD4+ T cells.

According to other specific embodiments, the T cells are CD8+ T cells.

According to specific embodiments, the T cells are memory T cells. Non-limiting examples of memory T cells include effector memory CD4+ T cells with a CD3+/CD4+/CD45RA−/CCR7− phenotype, central memory CD4+ T cells with a CD3+/CD4+/CD45RA−/CCR7+ phenotype, effector memory CD8+ T cells with a CD3+/CD8+ CD45RA−/CCR7−phenotype and central memory CD8+ T cells with a CD3+/CD8+ CD45RA−/CCR7+ phenotype.

As used herein the term “B cells” refers to a lymphocyte with a B cell receptor (BCR)+, CD19+ and or B220+ phenotype. B cells are characterized by their ability to bind a specific antigen and elicit a humoral response.

As used herein the term “NK cells” refers to differentiated lymphocytes with a CD16+ CD56+ and/or CD57+ TCR-phenotype. NK are characterized by their ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

As used herein the term “NKT cells” refers to a specialized population of T cells that express a semi-invariant αβ T-cell receptor, but also express a variety of molecular markers that are typically associated with NK cells, such as NK1.1. NKT cells include NK1.1⁺ and NK1.1⁻, as well as CD4⁺, CD4⁻, CD8⁺ and CD8⁻ cells. The TCR on NKT cells is unique in that it recognizes glycolipid antigens presented by the MHC I-like molecule CD1d. NKT cells can have either protective or deleterious effects due to their abilities to produce cytokines that promote either inflammation or immune tolerance.

According to specific embodiments the MNBCs obtained according to the methods of the invention do not comprise CD34+ hematopoietic stem/progenitor cells.

The cells of the present invention are obtained by administering a peptide having an amino acid sequence as set forth in SEQ ID NO:1 or an analog or derivative thereof to the subject.

As used herein the term “subject” refers to a mammal, e.g., human being at any age or of any gender.

According to specific embodiments, the subject is a donor subject (syngeneic or non-syngeneic e.g., allogeneic). Accordingly, and to some embodiments of the invention the subject is a healthy subject.

According to other specific embodiments, the subject is a recipient suffering from a pathology e.g. cancer. In a specific embodiment, this term encompasses individuals who are at risk to develop the pathology.

As used herein, the term “peptide” encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells.

According to a specific embodiment, the peptide is no more than 100 amino acids in length. According to a specific embodiment, the peptide is 5-100 amino acids in length. According to a specific embodiment, the peptide is 5-50 amino acids in length. According to a specific embodiment, the peptide is 5-20 amino acids in length. According to a specific embodiment, the peptide is 5-15 amino acids in length. According to a specific embodiment, the peptide is 10-20 amino acids in length. According to a specific embodiment, the peptide is 10-15 amino acids in length.

As used herein the term “peptide having an amino acid sequence as set forth in SEQ ID NO:1 or an analog or derivative thereof” refers to 4F-benzoyl-TN14003 (SEQ ID NO: 1, also known as BKT140, hereinafter BL-8040) peptide and functional analogs or derivatives thereof. The peptides of the present invention are structurally and functionally related to the peptides disclosed in patent applications WO 2002/020561 and WO 2004/020462, also known as “T-140 analogs”, as detailed hereinbelow. The peptide of the present invention is a CXCR4-antagnoistic peptide i.e. it reduces CXCR-4 activation by at least 10% as compared to same in the absence of the peptide antagonist. According to a specific embodiment the peptide antagonist is a competitive inhibitor. According to a specific embodiment the peptide antagonist is a non-competitive inhibitor.

A functional peptide, as used herein, is capable of elevating peripheral blood levels of at least one type of mononuclear blood cells (MNBCs) including, but not limited to, T cells, B cells, NK cells, NKT cells and immature DCs in a subject upon administration.

In various particular embodiments, the peptide analog or derivative has an amino acid sequence as set forth in the following formula (I) or a salt thereof:

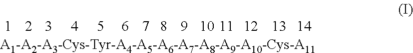

wherein:

A₁ is an arginine, lysine, ornithine, citrulline, alanine or glutamic acid residue or a N-α-substituted derivative of these amino acids, or A₁ is absent;

A₂ represents an arginine or glutamic acid residue if A₁is present, or A₂ represents an arginine or glutamic acid residue or a N-α-substituted derivative of these amino acids if A₁ is absent;

A₃ represents an aromatic amino acid residue;

A₄, A₅ and A₉ each independently represents an arginine, lysine, ornithine, citrulline, alanine or glutamic acid residue;

A₆ represents a proline, glycine, ornithine, lysine, alanine, citrulline, arginine or glutamic acid residue;

A₇ represents a proline, glycine, ornithine, lysine, alanine, citrulline or arginine residue;

A₈ represents a tyrosine, phenylalanine, alanine, naphthylalanine, citrulline or glutamic acid residue;

A₁₀ represents a citrulline, glutamic acid, arginine or lysine residue;

A₁₁ represents an arginine, glutamic acid, lysine or citrulline residue wherein the C-terminal carboxyl may be derivatized;

and the cysteine residue of the 4-position or the 13-position can form a disulfide bond, and the amino acids can be of either L or D form.

Exemplary peptides according to formula (I) are peptides having an amino acid sequence as set forth in any one of SEQ ID NOS:1-72, as presented in Table 1 hereinbelow.

TABLE 1
T-140 and currently preferred T-140 analogs

| | SEQ | |
|---|---|---|
| | ID | |
| Analog | NO: | Amino acid sequence |
| 4F-benzoyl- | 1 | 4F-benzoyl-Arg-Arg-Nal-Cys-Tyr-Cit-Lys-DLys-Pro-Tyr-Arg-Cit-Cys-Arg-NH₂ |
| TN14003 | | |
| AcTC14003 | 2 | Ac-Arg-Arg-Nal-Cys-Tyr-Cit-Lys-DLys-Pro-Tyr-Arg-Cit-Cys-Arg-OH |
| AcTC14005 | 3 | Ac-Arg-Arg-Nal-Cys-Tyr-Arg-Lys-DCit-Pro-Tyr-Arg-Cit-Cys-Arg-OH |
| AcTC14011 | 4 | Ac-Arg-Arg-Nal-Cys-Tyr-Cit-Lys-DCit-Pro-Tyr-Arg-Cit-Cys-Arg-OH |
| AcTC14013 | 5 | Ac-Arg-Arg-Nal-Cys-Tyr-Cit-Lys-DLys-Pro-Tyr-Cit-Cit-Cys-Arg-OH |
| AcTC14015 | 6 | Ac-Cit-Arg-Nal-Cys-Tyr-Cit-Lys-DLys-Pro-Tyr-Arg-Cit-Cys-Arg-OH |
| AcTC14017 | 7 | Ac-Cit-Arg-Nal-Cys-Tyr-Arg-Lys-DCit-Pro-Tyr-Arg-Cit-Cys-Arg-OH |
| AcTC14019 | 8 | Ac-Arg-Arg-Nal-Cys-Tyr-Arg-Lys-DCit-Pro-Tyr-Cit-Cit-Cys-Arg-OH |
| AcTC14021 | 9 | Ac-Cit-Arg-Nal-Cys-Tyr-Arg-Lys-DLys-Pro-Tyr-Cit-Cit-Cys-Arg-OH |
| AcTC14012 | 10 | Ac-Arg-Arg-Nal-Cys-Tyr-Cit-Lys-DCit-Pro-Tyr-Arg-Cit-Cys-Arg-NH₂ |
| AcTC14014 | 11 | Ac-Arg-Arg-Nal-Cys-Tyr-Cit-Lys-DLys-Pro-Tyr-Cit-Cit-Cys-Arg-NH₂ |
| AcTC14016 | 12 | Ac-Cit-Arg-Nal-Cys-Tyr-Cit-Lys-DLys-Pro-Tyr-Arg-Cit-Cys-Arg-NH₂ |
| AcTC14018 | 13 | Ac-Cit-Arg-Nal-Cys-Tyr-Arg-Lys-DCit-Pro-Tyr-Arg-Cit-Cys-Arg-NH₂ |
| AcTC14020 | 14 | Ac-Arg-Arg-Nal-Cys-Tyr-Arg-Lys-DCit-Pro-Tyr-Cit-Cit-Cys-Arg-NH₂ |
| AcTC14022 | 15 | Ac-Cit-Arg-Nal-Cys-Tyr-Arg-Lys-DLys-Pro-Tyr-Cit-Cit-Cys-Arg-NH₂ |
| TE14001 | 16 | H-DGlu-Arg-Nal-Cys-Tyr-Arg-Lys-DLys-Pro-Tyr-Arg-Cit-Cys-Arg-OH |
| TE14002 | 17 | H-Arg-Glu-Nal-Cys-Tyr-Arg-Lys-DLys-Pro-Tyr-Arg-Cit-Cys-Arg-OH |
| TE14003 | 18 | H-Arg-Arg-Nal-Cys-Tyr-Glu-Lys-DLys-Pro-Tyr-Arg-Cit-Cys-Arg-OH |
| TE14004 | 19 | H-Arg-Arg-Nal-Cys-Tyr-Arg-Glu-DLys-Pro-Tyr-Arg-Cit-Cys-Arg-OH |
| TE14005 | 20 | H-Arg-Arg-Nal-Cys-Tyr-Arg-Lys-DGlu-Pro-Tyr-Arg-Cit-Cys-Arg-OH |
| TE14006 | 21 | H-Arg-Arg-Nal-Cys-Tyr-Arg-Lys-DLys-Pro-Tyr-Glu-Cit-Cys-Arg-OH |
| TE14007 | 22 | H-Arg-Arg-Nal-Cys-Tyr-Arg-Lys-DLys-Pro-Tyr-Arg-Cit-Cys-Glu-OH |
| TE14011 | 23 | H-Arg-Arg-Nal-Cys-Tyr-Cit-Lys-DGlu-Pro-Tyr-Arg-Cit-Cys-Arg-NH₂ |
| TE14012 | 24 | H-Arg-Arg-Nal-Cys-Tyr-DGlu-Lys-DCit-Pro-Tyr-Arg-Cit-Cys-Arg-NH₂ |
| TE14013 | 25 | H-Arg-Arg-Nal-Cys-Tyr-DGlu-Lys-DGlu-Pro-Tyr-Arg-Cit-Cys-Arg-NH₂ |
| TE14014 | 26 | H-DGlu-Arg-Nal-Cys-Tyr-Cit-Lys-DGlu-Pro-Tyr-Arg-Cit-Cys-Arg-NH₂ |
| TE14015 | 27 | H-Arg-Arg-Nal-Cys-Tyr-Cit-Lys-DGlu-Pro-DGlu-Arg-Cit-Cys-Arg-NH₂ |
| TE14016 | 28 | H-Arg-Arg-Nal-Cys-Tyr-Cit-Lys-DGlu-Pro-Tyr-Arg-DGlu-Cys-Arg-NH₂ |
| AcTE14014 | 29 | Ac-DGlu-Arg-Nal-Cys-Tyr-Cit-Lys-DGlu-Pro-Tyr-Arg-Cit-Cys-Arg-NH₂ |
| AcTE14015 | 30 | Ac-Arg-Arg-Nal-Cys-Tyr-Cit-Lys-DGlu-Pro-DGlu-Arg-Cit-Cys-Arg-NH₂ |
| AcTE14016 | 31 | Ac-Arg-Arg-Nal-Cys-Tyr-Cit-Lys-DGlu-Pro-Tyr-Arg-DGlu-Cys-Arg-NH₂ |
| TF1: AcTE14011 | 32 | Ac-Arg-Arg-Nal-Cys-Tyr-Cit-Lys-DGlu-Pro-Tyr-Arg-Cit-Cys-Arg-NH₂ |
| TF2: guanyl- | 33 | guanyl-Arg-Arg-Nal-Cys-Tyr-Cit-Lys-DGlu-Pro-Tyr-Arg-Cit-Cys-Arg-NH₂ |
| TE14011 | | |
| TF3: TMguanyl- | 34 | TMguanyl-Arg-Arg-Nal-Cys-Tyr-Cit-Lys-DGlu-Pro-Tyr-Arg-Cit-Cys-Arg-NH₂ |
| TE14011 | | |
| TF4: TMguanyl- | 35 | TMguanyl-Arg-Nal-Cys-Tyr-Cit-Lys-DGlu-Pro-Tyr-Arg-Cit-Cys-Arg-NH₂ |
| TE14011 (2-14) | | |
| TF5: 4F-benzoyl- | 36 | 4F-benzoyl-Arg-Arg-Nal-Cys-Tyr-Cit-Lys-DGlu-Pro-Tyr-Arg-Cit-Cys-Arg-NH₂ |
| TE14011 | | |
| TF6: 2F-benzoyl- | 37 | 2F-benzoyl-Arg-Arg-Nal-Cys-Tyr-Cit-Lys-DGlu-Pro-Tyr-Arg-Cit-Cys-Arg-NH₂ |
| TE14011 | | |
| TF7: APA- | 38 | APA-Arg-Nal-Cys-Tyr-Cit-Lys-DGlu-Pro-Tyr-Arg-Cit-Cys-Arg-NH₂ |
| TE14011 (2-14) | | |
| TF8: desamino- | 39 | desamino-R-Arg-Nal-Cys-Tyr-Cit-Lys-DGlu-Pro-Tyr-Arg-Cit-Cys-Arg-NH₂ |
| R-TE14011 (2- | | |
| 14) | | |
| TF9: guanyl- | 40 | Guanyl-Arg-Nal-Cys-Tyr-Cit-Lys-DGlu-Pro-Tyr-Arg-Cit-Cys-Arg-NH₂ |
| TE14011 (2-14) | | |
| TF10: succinyl- | 41 | succinyl-Arg-Nal-Cys-Tyr-Cit-Lys-DGlu-Pro-Tyr-Arg-Cit-Cys-Arg-NH₂ |
| TE14011 (2-14) | | |
| TF11: glutaryl- | 42 | glutaryl-Arg-Nal-Cys-Tyr-Cit-Lys-DGlu-Pro-Tyr-Arg-Cit-Cys-Arg-NH₂ |
| TE14011 (2-14) | | |
| TF12: | 43 | deaminoTMG-APA-Arg-Nal-Cys-Tyr-Cit-Lys-DGlu-Pro-Tyr-Arg-Cit-Cys-Arg-NH₂ |
| deaminoTMG- | | |
| APA-TE14011 | | |
| (2-14) | | |
| TF15: H-Arg- | 44 | R-CH2-Arg-Nal-Cys-Tyr-Cit-Lys-DGlu-Pro-Tyr-Arg-Cit-Cys-Arg-NH₂ |
| CH2NH- | | |
| RTE14011 (2-14) | | |
| TF17: TE14011 | 45 | H-Arg-Nal-Cys-Tyr-Cit-Lys-DGlu-Pro-Tyr-Arg-Cit-Cys-Arg-NH₂ |
| (2-14) | | |
| TF18: TMguanyl- | 46 | TMguanyl-Arg-Arg-Nal-Cys-Tyr-Cit-Lys-DCit-Pro-Tyr-Arg-Cit-Cys-Arg-NH₂ |
| TC14012 | | |
| TF19: ACA- | 47 | ACA-Arg-Arg-Nal-Cys-Tyr-Cit-Lys-DCit-Pro-Tyr-Arg-Cit-Cys-Arg-NH₂ |
| TC14012 | | |
| TF20: ACA-T140 | 48 | ACA-Arg-Arg-Nal-Cys-Tyr-Arg-Lys-DLys-Pro-Tyr-Arg-Cit-Cys-Arg-OH |
| TZ14011 | 49 | H-Arg-Arg-Nal-Cys-Tyr-Cit-Arg-DLys-Pro-Tyr-Arg-Cit-Cys-Arg-NH₂ |
| AcTZ14011 | 50 | Ac-Arg-Arg-Nal-Cys-Tyr-Cit-Arg-DLys-Pro-Tyr-Arg-Cit-Cys-Arg-NH₂ |
| AcTN14003 | 51 | Ac-Arg-Arg-Nal-Cys-Tyr-Cit-Lys-DLys-Pro-Tyr-Arg-Cit-Cys-Arg-NH₂ |
| AcTN14005 | 52 | Ac-Arg-Arg-Nal-Cys-Tyr-Arg-Lys-DCit-Pro-Tyr-Arg-Cit-Cys-Arg-NH₂ |
| 4F-benzoyl- | 53 | 4F-benzoyl-Arg-Arg-Nal-Cys-Tyr-Cit-Lys-DGlu-Pro-Tyr-Arg-Cit-Cys-Arg-NHMe |
| TN14011-Me | | |
| 4F-benzoyl- | 54 | 4F-benzoyl-Arg-Arg-Nal-Cys-Tyr-Cit-Lys-DGlu-Pro-Tyr-Arg-Cit-Cys-Arg-NHEt |
| TN14011-Et | | |
| 4F-benzoyl- | 55 | 4F-benzoyl-Arg-Arg-Nal-Cys-Tyr-Cit-Lys-DGlu-Pro-Tyr-Arg-Cit-Cys-Arg-NHiPr |
| TN14011-iPr | | |
| 4F-benzoyl- | 56 | 4F-benzoyl-Arg-Arg-Nal-Cys-Tyr-Cit-Lys-DGlu-Pro-Tyr-Arg-Cit-Cys-Arg-tyramine |
| TN14011- | | |
| tyramine | | |
| TA14001 | 57 | H-Ala-Arg-Nal-Cys-Tyr-Arg-Lys-DLys-Pro-Tyr-Arg-Cit-Cys-Arg-OH |
| TA14005 | 58 | H-Arg-Arg-Nal-Cys-Tyr-Ala-Lys-DLys-Pro-Tyr-Arg-Cit-Cys-Arg-OH |
| TA14006 | 59 | H-Arg-Arg-Nal-Cys-Tyr-Arg-Ala-DLys-Pro-Tyr-Arg-Cit-Cys-Arg-OH |
| TA14007 | 60 | H-Arg-Arg-Nal-Cys-Tyr-Arg-Lys-DAla-Pro-Tyr-Arg-Cit-Cys-Arg-OH |
| TA14008 | 61 | H-Arg-Arg-Nal-Cys-Tyr-Arg-Lys-DLys-Ala-Tyr-Arg-Cit-Cys-Arg-OH |
| TA14009 | 62 | H-Arg-Arg-Nal-Cys-Tyr-Arg-Lys-DLys-Pro-Ala-Arg-Cit-Cys-Arg-OH |
| TA14010 | 63 | H-Arg-Arg-Nal-Cys-Tyr-Arg-Lys-DLys-Pro-Tyr-Ala-Cit-Cys-Arg-OH |
| TC14001 | 64 | H-Cit-Arg-Nal-Cys-Tyr-Arg-Lys-DLys-Pro-Tyr-Arg-Cit-Cys-Arg-OH |
| TC14003 | 65 | H-Arg-Arg-Nal-Cys-Tyr-Cit-Lys-DLys-Pro-Tyr-Arg-Cit-Cys-Arg-OH |
| TN14003 | 66 | H-Arg-Arg-Nal-Cys-Tyr-Cit-Lys-DLys-Pro-Tyr-Arg-Cit-Cys-Arg-NH₂ |
| TC14004 | 67 | H-Arg-Arg-Nal-Cys-Tyr-Arg-Cit-DLys-Pro-Tyr-Arg-Cit-Cys-Arg-OH |
| TC14012 | 68 | H-Arg-Arg-Nal-Cys-Tyr-Cit-Lys-DCit-Pro-Tyr-Arg-Cit-Cys-Arg-NH₂ |
| T-140 | 69 | H-Arg-Arg-Nal-Cys-Tyr-Arg-Lys-DLys-Pro-Tyr-Arg-Cit-Cys-Arg-OH |
| TC14011 | 70 | H-Arg-Arg-Nal-Cys-Tyr-Cit-Lys-DCit-Pro-Tyr-Arg-Cit-Cys-Arg-OH |
| TC14005 | 71 | H-Arg-Arg-Nal-Cys-Tyr-Arg-Lys-DCit-Pro-Tyr-Arg-Cit-Cys-Arg-OH |
| TC14018 | 72 | H-Cit-Arg-Nal-Cys-Tyr-Arg-Lys-DCit-Pro-Tyr-Arg-Cit-Cys-Arg-NH₂ |

According to a specific embodiment, in each one of SEQ ID NOs: 1-72, two cysteine residues are coupled in a disulfide bond.

In another embodiment, the analog or derivative has an amino acid sequence as set forth in SEQ ID NO:65 (H-Arg-Arg-Nal-Cys-Tyr-Cit-Lys-DLys-Pro-Tyr-Arg-Cit-Cys-Arg-OH; TC14003).

In another embodiment, the peptide used in the compositions and methods of the invention consists essentially of an amino acid sequence as set forth in SEQ ID NO: 1. In another embodiment, the peptide used in the compositions and methods of the invention comprises an amino acid sequence as set forth in SEQ ID NO: 1. In another embodiment, the peptide is at least 60%, at least 70% or at least 80% homologous to SEQ ID NO: 1. In another embodiment, the peptide is at least 90% homologous to SEQ ID NO: 1. In another embodiment, the peptide is at least about 95% homologous to SEQ ID NO: 1. Each possibility represents a separate embodiment of the present invention.

In various other embodiments, the peptide is selected from SEQ ID NOs: 1-72, wherein each possibility represents a separate embodiment of the present invention.

In another embodiment, the peptide has an amino acid sequence as set forth in any one of SEQ ID NOs: 1-4, 10, 46, 47, 51-56, 65, 66, 68, 70 and 71. In another embodiment, the peptide has an amino acid sequence as set forth in any one of SEQ ID NOs: 4, 10, 46, 47, 68 and 70. In another embodiment, the peptide has an amino acid sequence as set forth in any one of SEQ ID NOs: 1, 2, 51, 65 and 66. In another embodiment, the peptide has an amino acid sequence as set forth in any one of SEQ ID NOs: 53-56.

In an embodiment, the peptide has an amino acid sequence as set forth in SEQ ID NO: 1.

According to a specific embodiment, the peptide is as set forth in SEQ ID NO: 1. In another embodiment, the peptide has an amino acid sequence as set forth in SEQ ID NO: 2. In another embodiment, the peptide has an amino acid sequence as set forth in SEQ ID NO: 51. In another embodiment, the peptide has an amino acid sequence as set forth in SEQ ID NO: 66.

The peptides of some embodiments of the invention may be synthesized by any techniques that are known to those skilled in the art of peptide synthesis. For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing peptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final peptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide and so forth. Further description of peptide synthesis is disclosed in U.S. Pat. No. 6,472,505.

Large scale peptide synthesis is described by Andersson Biopolymers 2000;55(3):227-50.

According to specific embodiments, the peptide is administered to the subject in combination with one or more white blood cell mobilizing agents. For example, the peptide may be administered in sequential or concomitant combination with one or more other growth factors or cytokines that affect mobilization such as, but not limited to, G-CSF, GM-CSF and SCF.

As mentioned, following administration, peripheral blood is collected.

As used herein the term “collecting a peripheral blood” refers to the process in which peripheral blood is retrieved from the subject. Exemplary time ranges for collecting the peripheral blood from the subject following administration of the peptide include but are not limited to 4-48 hours, 4-24 hours, 4-12 hours, 4-8 hours, 4-6 hours, 6-8 hours, 4-5 hours, 5-6 or 3-4 hours.

According to specific embodiment collecting is effected 4-48 hours following peptide administering.

According to specific embodiment collecting is effected 4-8 hours following peptide administering.

According to specific embodiments, peripheral blood collection is effected more than once in a period of up to 48 hours following peptide administration.

According to specific embodiments, for DCs collection exemplary time ranges for collecting the peripheral blood from the subject following administration of the peptide include but are not limited to 3-8 hours, 4-8 hours, 3-6 hours, 4-6 hours, 3-5 hours, 3-4 hours and 4-5 hours.

Methods of collecting peripheral blood are well known in the art and include, but not limited to drawing of up to 500 ml whole blood from the subject and collection in a container containing an anti-coagulant (e.g. heparin or citrate); and apheresis.

As used herein, the term “apheresis” refers to a procedure in which the peripheral blood of an individual is passed through an apparatus, yielding a predominant constituent (e.g. mononuclear cells), and returning the other constituents to the subject's circulation. Apheresis is in general a three-step process comprising: (1) withdrawing blood from the subject, (2) separating the blood components (e.g. based on density), and (3) returning certain component(s) of the blood to the subject by transfusion. The blood is normally separated into three fractions: red blood cells (about 45% of total blood), “buffy coat’ (less than 1% of total blood) and plasma (about 55% of total blood). Various types of apheresis procedures can be used depending on the component of blood that is being removed. For example, “plasmapheresis” refers to the separation and collection of blood plasma, “thrombocytapheresis” refers to the separation and collection of platelets, while “leukapheresis” refers to the separation and collection of leukocytes, “granulocytapheresis” refers to the separation and collection of granulocytes (neutrophils, eosinophils, and basophils); and “lymphocytapheresis” refers to the separation and collection of lymphocytes.

According to a specific embodiment collecting the peripheral blood is effected by leukapheresis.

The mobilization of several types of MNBCs (e.g. T cells) following peptide administration is very rapid and continues for about 48 hours before the number of cells in the periphery starts to decline. Thus, first collection of peripheral blood can be effected already about 4 hours following peptide administration allowing for additional step(s) of collection if needed. Thus, according to specific embodiments, the collection of peripheral of the subject may be repeated at least once, at least twice, no later than 48 hours following peptide administration.

According to specific embodiments the MNBCs are further purified from the peripheral blood. Methods of purifying cells are well known in the art (e.g., see above apheresis and further below).

As the peptides of the present invention induce migration of MNBCs to the peripheral blood, the cells collected according to the method can be further used for adoptive transfer therapies or for the development of vaccines for the treatment of several diseases such as, but not limited to cancer, infectious disease, autoimmune disease, allergy and graft rejection. Methods of developing vaccines and cells for adoptive transfer therapies are well known in the art (see e.g. Hildebrandt et al. Cytotherapy. 2014 16(40): S120-S129; Leen et al. Immunol Rev. 2014; 258(1): 12-29; Qian et al. Journal of Immunology Research Volume 2014, Article ID 525913, 9 pages; Martelli et al. Blood. 2014;123(7):967-973; Ophir and Reisner Front Immunol. 2012 3:93; Lask et al. Blood. 2013;121(15):3033-3040; Palucka and Banchereau Immunity. 2013; 39(1): 38-48; Pizzurro and Barrio Front Immunol. 2015 Mar. 3; 6:91; Ludewig, Curr Top Microbiol Immunol. 2003;276:199-214; Miller and Bhardwaj, Expert Opin. Biol. Ther. (2014) 14(11):1545-1549; Zhou et al. Immunology, 2012 136, 385-396; Raïch-Regúet al. Immunology Letters 161 (2014) 216-221; Vassalli Journal of Transplantation Volume 2013, Article ID 761429, 17 pages; Galluzzi et al. Oncoimmunology. 2012; 1(3):306-315; Itzhaki et al. Immunotherapy. 2013; 5(1):79-90; Rosenberg et al. Clinical cancer research: an official journal of the American Association for Cancer Research. 2011; 17(13):4550-4557; Bouquie' et al. Cancer Immunol Immunother. 2009;58:553-66; Lu et al. Journal of immunology. 2013; 190(12):6034-6042; Robbins et al. Journal of clinical oncology: official journal of the American Society of Clinical Oncology. 2011; 29(7):917-924; Palucka K and Banchereau, Nature reviews Cancer. 2012; 12(4):265-277; and Shtivelman et al. Oncotarget. 2014 Apr. 15; 5(7):1701-52; the contents of each of which is fully incorporated herein by reference) and are further described hereinbelow.

Thus, according to specific embodiment, following collection of a peripheral blood of a subject, the method further comprises enriching at least one type MNBCs effective for the treatment of the indicated disease.

As used herein, the term “enriching” refers to the process of increasing the percentage of a specific cell type as compared to its percentage in the biological sample prior to said enriching. According to specific embodiment the increase is by at least 5%. According to other specific embodiments the increase is by at least 10%, by at least 20%, by at least 30%, by at least 40%, by at least 50%, by at least 60%, by at least 70%, by at least 80%, by at least 90% or more say by at least 99%.

Methods of enriching cell populations are well known in the art and include purification (e.g., by size, density), activation, expansion, promotion of presentation of a specific antigen and promotion of presentation of a specific receptor and combinations thereof.

According to a specific embodiment, the step of enriching is effected ex vivo.

As used herein, the terms “purification”, “selection” and “isolation” are interchangeably used and refer to the collection of a specific cell type from the peripheral blood while discarding other(s). According to specific embodiments, the purification process results in a population of cells in which at least about 75%, at least about 85%, at least about 90%, at least 95% pure, or at least about 99% of the cells are the cells of interest (e.g., T cells or DCs).

According to a specific embodiment, enriching comprises purifying at least one type of MNBCs selected from the group consisting of DCs, T cells, B cells, NK cells and NKT cells from said peripheral blood following said collecting.

According to specific embodiments, the purification comprises discarding CD34+ hematopoietic stem cells from the collected peripheral blood.

According to other specific embodiments, the purification comprises discarding platelets from the collected peripheral blood.

There are several methods and reagents known to those skilled in the art for purifying MNBCs from whole blood such as leukapheresis, sedimentation, density gradient centrifugation, centrifugal elutriation, fractionation, chemical lysis of e.g. red blood cells and selection of specific cell types using cell surface markers.

Thus, for example, MNBCs can be isolated by overlaying the blood onto a reagent, such as ficoll, for gradient separation and then centrifuging thereby localizing the majority of the MNBCs in a buffy interface layer which can be harvested.

Another way to isolate MNBCs is to extract the cells from whole blood using hypotonic lysis such as ACK which will preferentially lyse red blood cells.

According to specific embodiments, the cells can be purified differentially based on surface antigens expressed by certain types of DCs, T cells, NK cells and NKT cells e.g. using FACS sorter or magnetic cell separation techniques.

Purification can also be effected by depletion of specific cell types, thus for example depletion of T cells, e.g. CD3+, CD2+, TCRα/β+, CD4+ and/or CD8+ cells, or B cells, e.g. CD19+ and/or CD20+ cells, may be carried out using methods known in the art, such as by eradication (e.g. killing) with specific antibodies or by affinity based purification based on negative selection e.g. such as by the use of magnetic cell separation techniques, FACS sorter and/or capture ELISA labeling.

Such methods are described herein and in The Handbook Of Experimental Immunology, Volumes 1 to 4, (D. N. Weir, editor) and Flow Cytometry And Cell Sorting (A. Radbruch, editor, Springer Verlag, 2000). For example, cells can be sorted by, fluorescence activated cell sorting (FACS).

Other methods for cell sorting include, for example, panning and separation using affinity techniques, including those techniques using solid supports such as plates, beads and columns. Thus, biological samples may be separated by “panning” with an antibody attached to a solid matrix, e.g. to a plate.

Alternatively, cells may be sorted/separated by magnetic separation techniques, and some of these methods utilize magnetic beads. Different magnetic beads are available from a number of sources, including for example, Myltenni Biotech (Teterow, Germany), Dynal (Norway), Advanced Magnetics (Cambridge, Mass., U.S.A.), Immuncon (Philadelphia, U.S.A.), Immunotec (Marseille, France), Invitrogen, Stem cell Technologies (U.S.A) and Cellpro (U.S.A). Alternatively, antibodies can be biotinylated or conjugated with digoxigenin and used in conjunction with avidin or anti-digoxigenin coated affinity columns. During the separation process measures are taken to maintain viability and functionality of the cells.

According to an embodiment, different depletion/separation methods can be combined, for example, magnetic cell sorting can be combined with FACS, to increase the separation quality or to allow sorting by multiple parameters.

According to specific embodiments the purification is effected by a method selected from the group consisting of FACS and magnetic bead separation.

A known method of enriching a cell population is by activating the specifically desired population of cells.

As used herein the term “activation” refers to the process of stimulating an MNBC (e.g. T cell) that results in cellular proliferation, maturation, cytokine production and/or induction of regulatory or effector functions.

According to specific embodiments activating comprises contacting said peripheral blood or a purified population of cells thereof (as described above) with a co-stimulatory molecule.

As used herein the term “co-stimulatory molecule” refers to a molecule that positively regulates an immune cell activation or function by transmitting a stimulatory signal or suppressing an inhibitory signal resulting in activation of the immune cell. According to specific embodiments, the co-stimulatory molecule is selected form the group consisting of an immune check point regulator, LPS and TLR ligand.

As used herein, the term “LPS” refers to Lipopolysaccharides, also known as lipoglycans. LPS are large molecules consisting of a lipid and a polysaccharide joined by a covalent bond; they are found in the outer membrane of Gram-negative bacteria, act as endotoxins and activate immune cells.

As used herein, the term “TLR ligands” refers to ligands that activate toll-like receptors (TLRs) which are membrane-spanning, non-catalytic receptors that recognize structurally conserved molecules derived from microbes. Binding of a TLR ligand to TLR receptor induces activation of immune cells.

As used herein the term “immune-check point regulator” refers to a molecule that modulates the activity of one or more immune-check point proteins in an agonistic or antagonistic manner resulting in activation of an immune cell.

As used herein the term “immune-check point protein” refers to a protein that regulates an immune cell activation or function. Immune check-point proteins can be either co-stimulatory proteins (i.e. transmitting a stimulatory signal resulting in activation of an immune cell) or inhibitory proteins (i.e. transmitting an inhibitory signal resulting in suppressing activity of an immune cell). According to specific embodiment, the immune check point protein regulates activation or function of a T cell. Numerous checkpoint proteins are known in the art and include, but not limited to, PD1, PDL-1, B7H2, B7H4, CTLA-4, CD80, CD86, LAG-3, TIM-3, KIR, IDO, CD19, OX40, 4-1BB (CD137), CD27, CD70, CD40, GITR, CD28 and ICOS (CD278).

According to specific embodiments, the immune-check-point regulator is selected form the group consisting of anti-CTLA4, anti-PD-1, and CD40 agonist.

According to specific embodiments, the immune-check point regulator is selected form the group consisting of anti-CTLA4, anti-PD-1, anti-PDL-1, CD40 agonist, 4-1BB agonist, GITR agonist and OX40 agonist.

CTLA4 is a member of the immunoglobulin superfamily, which is expressed on the surface of Helper T cells and transmits an inhibitory signal to T cells upon ligand binding. As used herein, the term “anti-CTLA4” refers to an antagonistic molecule that binds CTLA4 (CD152) and suppresses its suppressive activity. Thus, an anti-CTLA4 prevents the transmission of the inhibitory signal and thereby acts as a co-stimulatory molecule. According to a specific embodiment, the anti-CDLA4 molecule is an antibody.

PD-1 (Programmed Death 1) is a member of the extended CD28/CTLA-4 family of T cell regulators which is expressed on the surface of activated T cells, B cells, and macrophages and transmits an inhibitory signal upon ligand binding. As used herein, the term “anti-PD1” refers to an antagonistic molecule that binds PD-1 and suppresses its suppressive activity. Thus, an anti-PD-1 prevents the transmission of the inhibitory signal and thereby acts as a co-stimulatory molecule. According to a specific embodiment, the anti-PD1 molecule is an antibody. Numerous anti-PD-1 antibodies are known in the art see e.g. Topalian, et al. NEJM 2012.

PDL-1 is a ligand of PD-1. Binding of PDL-1 to its receptor PD-1 transmits an inhibitory signal to the cell expressing the PD-1. As used herein, the tem “anti-PDL-1” refers to an antagonistic molecule that inhibits PD-1 signaling by binding to or inhibiting PD-L1 from binding and/or activating PD-1. Thus, an anti-PD-1 prevents the transmission of the inhibitory signal and thereby acts as a co-stimulatory molecule. According to specific embodiments, the anti-PD-L1 is an anti-PD-L1 antibody. Numerous anti-PDL-1 antibodies are known in the art see e.g. Brahmer, et al. NEJM 2012.

CD40 (CD154) is a co-stimulatory receptor found on antigen presenting cells and transmits an activation signal upon ligand binding. As used herein, the term “CD40 agonist” refers to an agonistic molecule that binds CD40 (CD154) and thereby induces activation of the antigen presenting cell.

OX40 belongs to the TNF receptor super family and leads to expansion of CD4+ and CD8+ T cells. As used herein, the term “OX40 agonist” refers to an agonistic molecule that binds and activates OX40.

GITR (glucocorticoid-induced tumor necrosis factor receptor) is a surface receptor molecule that has been shown to be involved in inhibiting the suppressive activity of T-regulatory cells and extending the survival of T-effector cells. As used herein, the term “GITR agonist” refers to an agonistic molecule that binds and activates GITR. According to a specific embodiment, the GITR agonist is an antibody.

As noted above another method of enriching a cell population is expanding the desired population of cells.

As used herein, the term “expansion” and “proliferation” are interchangeably used and refer to an increase in the number of cells in a population by means of cell division. Cell proliferation is generally understood to result from the coordinated activation of multiple signal transduction pathways in response to the environment, including growth factors and other mitogens. Cell proliferation may also be promoted by release from the actions of signals and mechanisms that block or negatively affect cell proliferation. Methods of inducing proliferation of MNBCs are well known in the art and include, but not limited to, contacting the cells with anti-CD3/CD28 antibody, anti-CD3/CD28 activation beads, Tetanus toxoid peptides, selected antigens presented in the context of antigen presenting cells, and cytokines capable of inducing proliferation of MNBCs.

According to specific embodiment, activating or expanding comprises contacting said peripheral blood or a purified population of cells thereof with a cytokine capable of inducing activation and/or proliferation of a T cell.

Non-limiting examples of cytokines capable of inducing activation and or proliferation of a T cells include, but are not limited to IL-2, IFNα, IL-12, IFN-gamma, TNF-a, IL-15, IL-6 and IL-1.

According to specific embodiments, the cytokine is selected from the group consisting of IL-2, IFNα and IL-12.

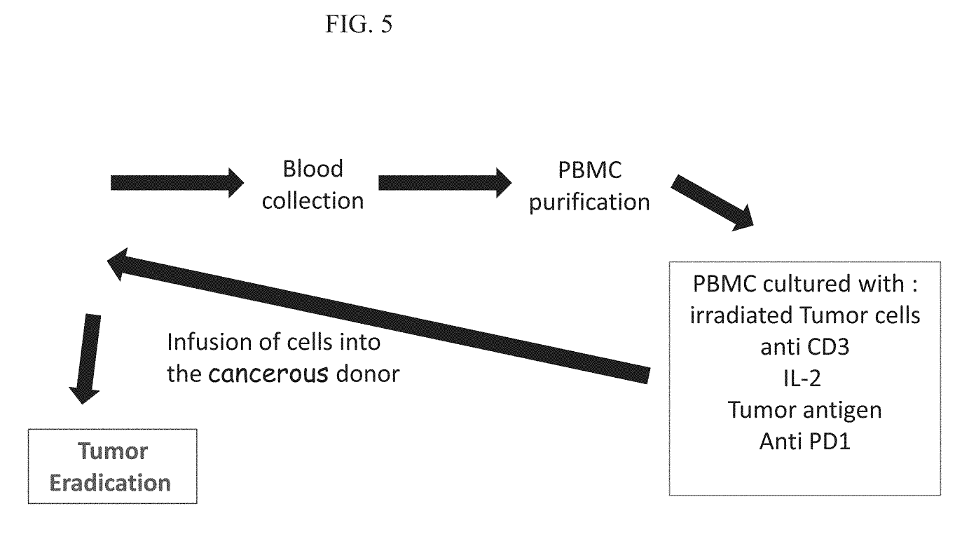

According to a specific embodiment, enriching for tumor specific T cells is effected in the presence of tumor antigens, cytokines anti CD3 and/or immune check point regulators (e.g., anti-PD1 or others, such as listed herein). It will be appreciated that administration of cytokines and/or check point regulators can also be effected following cell transplantation (see FIG. 5).

An additional method of enriching a specific cell population can be effected by promoting presentation of a specific antigen leading to enrichment of a population of antigen presenting cells presenting the specific antigen and/or activation and/or proliferation of antigen-specific population of cells (e.g. T cells).

As used herein, the term “promotion of presentation of a specific antigen” refers to inducing presentation of an antigen by an antigen presenting cell (APC) such as a DC. Promoting presentation of the antigen may be effected by contacting the peripheral blood or a purified population of cells thereof which contains APCs with the respective antigen, transducing an APC with mRNA of the respective antigen or contacting the peripheral blood or a purified population of cells thereof with an APC that presents the desired antigen.

Another method of enriching a specific cell population can be effected by promoting presentation of a specific receptor leading to enrichment of a population of antigen-specific population of cells (e.g. T cells, B cells).

As used herein, the term “promotion of presentation of specific receptor” refers to inducing expression of receptor, such as TCR or BCR, which recognizes a target antigen, typically by genetic engineering.

Example 4 of the Examples section which follows teaches enrichment for cells presenting anti-cancer antigens.

Following the enrichment, the specific population of cells effective to the treatment of the disease can be further subjected to purification.

Thus, according to a specific embodiment, the method further comprising purifying said at least one type of MNBCs following said enriching.

According to specific embodiments, the cells obtained according to the methods can be used freshly or stored e.g., cryopreserved (i.e. frozen) at e.g. liquid nitrogen temperature at any stage (e.g. following collection of the peripheral blood, following enrichment or following final purification) for long periods of time (e.g., months, years)for future use.

Methods of cryopreservation are commonly known by one of ordinary skill in the art and are disclosed e.g. in International Patent Application Publication Nos. WO2007054160 and WO 2001039594 and US Patent Application Publication No. US20120149108.

According to specific embodiments, the cells obtained according to the methods disclosed herein can be stored in a cell bank or a depository or storage facility.

The teachings described can be used to obtain cancer-reactive MNBCs that can be further used for adoptive transfer therapies or for the development of vaccines for the treatment of cancer.

Thus, according to an aspect of the present invention, there is provided a method of obtaining cells effective for the treatment of cancer, the method comprising:

(a) administering to a subject an effective amount of a peptide having an amino acid sequence as set forth in SEQ ID NO:1 or an analog or derivative thereof;

(b) collecting peripheral blood of said subject; and

(c) enriching from said peripheral blood at least one type of mononuclear blood cells (MNBCs) capable of eliciting an immune response against a cancerous cell,

thereby obtaining the cells effective for the treatment of cancer.

The terms “cancer” and “cancerous” refer to or describe the physiological condition in mammals that is typically characterized by unregulated cell growth. Cancers which can be treated by the method of this aspect of some embodiments of the invention can be any solid or non-solid cancer and/or cancer metastasis. Examples of cancer include but are not limited to, carcinoma, lymphoma, blastoma, sarcoma, and leukemia. More particular examples of such cancers include squamous cell cancer, lung cancer (including small-cell lung cancer, non-small-cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high-grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs'syndrome. Preferably, the cancer is selected from the group consisting of breast cancer, colorectal cancer, rectal cancer, non-small cell lung cancer, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, and multiple myeloma. The cancerous conditions amenable for treatment of the invention include metastatic cancers.

Enriching for an MNBC capable of eliciting an immune response against a cancerous cell can be effected by any method known in the art (see e.g. Hildebrandt et al. Cytotherapy. 2014 16(40): S120-S129; Leen et al. Immunol Rev. 2014; 258(1): 12-29; Qian et al. Journal of Immunology Research Volume 2014, Article ID 525913, 9 pages; Palucka and Banchereau Immunity. 2013; 39(1): 38-48; and Pizzurro and Barrio Front Immunol. 2015 Mar. 3; 6:91).

According to specific embodiments, enriching is effected by a method selected from the group consisting of:

(i) selecting anti-cancer reactive cells;

(ii) activating anti-cancer reactive cells;

(iii) expanding anti-cancer reactive cells;

(iv) promoting presentation of a cancer antigen; and

(v) promoting presentation of an anti-cancer receptor.

As used herein, the term “anti-cancer reactive cell” refers to a MNBC capable of eliciting an immune response (e.g. T cell, NK cell) against a cancerous cell.

According to specific embodiment, enriching comprises contacting said peripheral blood or a purified population of cells thereof with a cancer antigen selected from the group consisting of a cancer antigenic peptide or polypeptide, a cancer cell lysate, a cancerous cell and a DC presenting a cancer antigen.

Thus, for example, for generation of an anti-cancer DC vaccine, immature DCs comprised in the peripheral blood or a purified population of cells thereof are expanded ex-vivo and contacted with a cancer antigen or a cancer cell lysate to thereby induce presentation of the cancer antigen. Alternatively, promoting presentation of a cancer antigen by a DC comprises transfecting said DC with an mRNA coding for a cancer antigen. Non-limiting examples of cancer antigens are listed below.

For a DC based immunotherapy to be successful, a combination of immature and mature cells and their synergistic action may be useful. Immature DCs efficiently capture and process antigens, however antigen presentation by an immature DC usually results in immune tolerance (see e.g. Steinman et al., Annu Rev Immunol. 2003 21:685-711; and Tarbell et al., Exp Med. 2007; 204:191-201). During the maturation process, the DCs become less efficient in antigen capturing but more specialized in presenting immunogenic peptides and in activating naïve T cells.

Thus, according to specific embodiments, when said cells comprise immature DCs the method comprises inducing maturation of said immature DCs (e.g. following capture and presentation of the cancer antigen).

Exemplary maturation agents include tumor necrosis factor (TNF)-α, IL-1β, IL-6, prostaglandin E2, BCG, IFN-γ, LPS, CD40L, monophosphoryl lipid A (MPL), eritoran (CAS number 185955-34-4), virus infection and different adjuvants.

As used herein, the term “cancer antigen” refers to an antigen is overexpressed or solely expressed by a cancerous cell as compared to a non-cancerous cell. A cancer antigen may be a known cancer antigen or a new specific antigen that develops in a cancer cell (i.e. neoantigens).

Non-limiting examples for known cancer antigens include MAGE-AI, MAGE-A2, MAGE-A3, MAGE-A4, MAGE-AS, MAGE-A6, MAGE-A7, MAGE-AS, MAGE-A9, MAGE-AIO, MAGE-A11, MAGE-A12, GAGE-I, GAGE-2, GAGE-3, GAGE-4, GAGE-5, GAGE-6, GAGE-7, GAGE-8, BAGE-1, RAGE-1, LB33/MUM-1, PRAME, NAG, MAGE-Xp2 (MAGE-B2), MAGE-Xp3 (MAGE-B3), MAGE-Xp4 (MAGE-B4), MAGE-C1/CT7, MAGE-C2, NY-ES0-1, LAGE-1, SSX-1, SSX-2(HOM-MEL-40), SSX-3, SSX-4, SSX-5, SCP-1 and XAGE, melanocyte differentiation antigens, p53, ras, CEA, MUCI, PMSA, PSA, tyrosinase, Melan-A, MART-I, gp100, gp75, alphaactinin-4, Bcr-Ab1 fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAA0205, Mart2, Mum-2, and 3, neo-PAP, myosin class I, OS-9, pml-RAR alpha fusion protein, PTPRK, K-ras, N-ras, Triosephosphate isomerase, GnTV, Herv-K-mel, NA-88, SP17, and TRP2-Int2, (MART-I), E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p1SOerbB-3, c-met, nm-23H1, PSA, TAG-72-4, CA 19-9, CA 72-4, CAM 17.1, NuMa, K-ras, alpha.-fetoprotein, 13HCG, BCA225, BTAA, CA 125, CA 15-3 (CA 27.29\BCAA), CA 195, CA 242, CA-50, CAM43, CD68\KP1, CO-029, FGF-5, 0250, Ga733 (EpCAM), HTgp-175, M344, MA-50, MG7-Ag, MOV18, NB\170K, NYCO-I, RCASI, SDCCAG16, TA-90 (Mac-2 binding protein\cyclophilin C-associated protein), TAAL6, TAG72, TLP, TPS, tyrosinase related proteins, TRP-1, or TRP-2.

Other tumor antigens that may be expressed include out-of-frame peptide-MHC complexes generated by the non-AUG translation initiation mechanisms employed by “stressed” cancer cells (Malarkannan et al. Immunity 1999).

Other tumor antigens that may be expressed are well-known in the art (see for example W000/20581; Cancer Vaccines and Immunotherapy (2000) Eds Stern, Beverley and Carroll, Cambridge University Press, Cambridge). The sequences of these tumor antigens are readily available from public databases but are also found in WO 1992/020356 AI, WO 1994/005304 AI, WO 1994/023031 AI, WO 1995/020974 AI, WO 1995/023874 AI & WO 1996/026214 AI.

For EBV-associated lymphoma, EBV-specific antigens can be used as the cancer antigen.

Alternatively or additionally a tumor antigen may be identified using cancer cells obtained from the subject by e.g. biopsy. For example, a method as described herein may comprise the step of identifying a tumor antigen which is displayed by one or more cancer cells in a sample obtained from the subject.

As a non-limiting example, for the generation of T cells that can be used for adoptive T cells transfer for cancer therapy the peripheral blood or purified population thereof comprising CD3+, CD4+ or CD8+ T cells or tumor-associated lymphocytes (TALs) selected for T-cell receptor (TCR) specificity (see e.g. Cancer Immunol Immunother. 2009; 58: 553-66.) is activated and expanded ex-vivo by incubation with mature DCs preloaded with tumor antigens (e.g., soluble proteins) or transfected with mRNA coding for cancer antigens. The DCs used are purified according to the teachings described hereinabove or using any method well known in the art such as DCs derived from monocytes or CD34+ cells. Alternatively, the peripheral blood or a purified population thereof comprising both APCs (e.g. DCs) and T cells (e.g. CD3+, CD4+ or CD8+ T cells or TALs selected for TCR specificity) is contacted with tumor antigens (see e.g., Example 4 of the Examples section which follows, in which irradiated cancer cells are used as tumor antigens for the generation of cells useful for adoptive transfer therapy for the treatment of cancer) or transfected with mRNA coding for cancer antigens under conditions which allow capturing and presentation of the cancer antigen by the APC leading to activation and expansion of anti-cancer antigen-specific T cells.

Other methods of expanding and processing T cells for anti-cancer adoptive cell transfer include redirecting T cell specificity by promoting presentation of an anti-cancer receptor by way of transducing with a T cell receptor (TCR) or a chimeric antigen receptor (CAR).

As used herein “transduction with a TCR” refers to cloning of variable α- and β-chains are from T cells with specificity against a cancer antigen presented in the context of MHC. Method of transducing with a TCR are known in the art and are disclosed e.g. in Nicholson et al. Adv Hematol. 2012; 2012:404081; Wang and Rivière Cancer Gene Ther. 2015 March; 22(2):85-94); and Lamers et al, Cancer Gene Therapy (2002) 9, 613-623.

As used herein “transducing with a CAR” refers to cloning of a nucleic acid sequence encoding a chimeric antigen receptor (CAR), wherein the CAR comprises an antigen recognition moiety and a T-cell activation moiety. A chimeric antigen receptor (CAR) is an artificially constructed hybrid protein or polypeptide containing an antigen binding domain of an antibody (e.g., a single chain variable fragment (scFv)) linked to T-cell signaling or T-cell activation domains. Method of transducing with a CAR are known in the art and are disclosed e.g. in Davila et al. Oncoimmunology. 2012 Dec 1;1(9):1577-1583; Wang and Rivière Cancer Gene Ther. 2015 March; 22(2):85-94); and Maus et al. Blood. 2014 Apr. 24; 123(17):2625-35.

The teachings described herein are also relevant for obtaining cells effective for the treatment of infectious diseases.

Thus, according to another aspect of the present invention, there is provided a method of obtaining cells effective for the treatment of an infectious disease, the method comprising:

(a) administering to a subject an effective amount of a peptide having an amino acid sequence as set forth in SEQ ID NO:1 or an analog or derivative thereof;

(b) collecting peripheral blood of said subject; and

(c) obtaining from said peripheral blood at least one type of mononuclear blood cells (MNBCs) capable of eliciting an immune response against a pathogen, thereby obtaining the cells effective for the treatment of the infectious disease.

As used herein, the term “infectious disease” refers to a disease induced by a pathogen. Specific examples of pathogens include, viral pathogens, bacterial pathogens e.g., intracellular mycobacterial pathogens (such as, for example, Mycobacterium tuberculosis), intracellular bacterial pathogens (such as, for example, Listeria monocytogenes), or intracellular protozoan pathogens (such as, for example, Leishmania and Trypanosoma).

Specific types of viral pathogens causing infectious diseases treatable according to the teachings of the present invention include, but are not limited to, retroviruses, circoviruses, parvoviruses, papovaviruses, adenoviruses, herpesviruses, iridoviruses, poxviruses, hepadnaviruses, picornaviruses, caliciviruses, togaviruses, flaviviruses, reoviruses, orthomyxoviruses, paramyxoviruses, rhabdoviruses, bunyaviruses, coronaviruses, arenaviruses, and filoviruses.

Specific examples of viral infections which may be treated according to the teachings of the present invention include, but are not limited to, human immunodeficiency virus (HIV)-induced acquired immunodeficiency syndrome (AIDS), influenza, rhinoviral infection, viral meningitis, Epstein-Barr virus (EBV) infection, hepatitis A, B or C virus infection, measles, papilloma virus infection/warts, cytomegalovirus (CMV) infection, Herpes simplex virus infection, yellow fever, Ebola virus infection, rabies, etc.

Enriching for an MNBC capable of eliciting an immune response against a pathogen can be effected by any method known in the art (see e.g. Hildebrandt et al. Cytotherapy. 2014 16(40): S120-S129; Leen et al. Immunol Rev. 2014; 258(1): 12-29; Ludewig, Curr Top Microbiol Immunol. 2003;276:199-214; Miller and Bhardwaj, Expert Opin. Biol. Ther. (2014) 14(11):1545-1549; and Zhou et al. Immunology, 2012 136, 385-396).

According to specific embodiments, the cells are selected from the group consisting of memory T cells, pathogen-specific T cells and DCs presenting a pathogenic antigen.

The teachings described herein are also relevant for obtaining cells effective for the treatment of an autoimmune disease, allergy or graft rejection disease. Thus, according to another aspect of the present invention, there is provided a method of obtaining cells effective for treatment of an autoimmune disease, allergy or graft rejection disease, the method comprising:

(a) administering to a subject an effective amount of a peptide having an amino acid sequence as set forth in SEQ ID NO:1 or an analog or derivative thereof;

(b) collecting peripheral blood from said subject;

(c) obtaining from said peripheral blood at least one type of mononuclear blood cells (MNBCs) capable of inducing tolerance to an autoimmune cell an allergen or a graft;

thereby obtaining the cells effective for treatment of the autoimmune disease, allergy or graft rejection disease.

According to specific embodiments, the disease is an autoimmune disease. Specific examples of autoimmune diseases which may be treated according to the teachings of the present invention include, but are not limited to, rheumatoid diseases, rheumatoid autoimmune diseases, rheumatoid arthritis (Krenn V. et al., Histol Histopathol 2000 July; 15 (3):791), spondylitis, ankylosing spondylitis (Jan Voswinkel et al., Arthritis Res 2001; 3 (3): 189), systemic diseases, systemic autoimmune diseases, systemic lupus erythematosus (Erikson J. et al., Immunol Res 1998;17 (1-2):49), sclerosis, systemic sclerosis (Renaudineau Y. et al., Clin Diagn Lab Immunol. 1999 March; 6 (2):156); Chan O T. et al., Immunol Rev 1999 June; 169:107), glandular diseases, glandular autoimmune diseases, pancreatic autoimmune diseases, diabetes, Type I diabetes (Zimmet P. Diabetes Res Clin Pract 1996 October; 34 Suppl:S125), thyroid diseases, autoimmune thyroid diseases, Graves' disease (Orgiazzi J. Endocrinol Metab Clin North Am 2000 June; 29 (2):339), thyroiditis, spontaneous autoimmune thyroiditis (Braley-Mullen H. and Yu S, J Immunol 2000 Dec 15;165 (12):7262), Hashimoto's thyroiditis (Toyoda N. et al., Nippon Rinsho 1999 August; 57 (8):1810), myxedema, idiopathic myxedema (Mitsuma T. Nippon Rinsho. 1999 August; 57 (8):1759); autoimmune reproductive diseases, ovarian diseases, ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), repeated fetal loss (Tincani A. et al., Lupus 1998;7 Suppl 2:S107-9), neurodegenerative diseases, neurological diseases, neurological autoimmune diseases, multiple sclerosis (Cross A H. et al., J Neuroimmunol 2001 Jan. 1; 112 (1-2):1), Alzheimer's disease (Oron L. et al., J Neural Transm Suppl. 1997;49:77), myasthenia gravis (Infante A J. And Kraig E, Int Rev Immunol 1999;18 (1-2):83), motor neuropathies (Kornberg A J. J Clin Neurosci. 2000 May; 7 (3):191), Guillain-Barre syndrome, neuropathies and autoimmune neuropathies (Kusunoki S. Am J Med Sci. 2000 April; 319 (4):234), myasthenic diseases, Lambert-Eaton myasthenic syndrome (Takamori M. Am J Med Sci. 2000 April; 319 (4):204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) 2000 January; 156 (1):23); neuropathies, dysimmune neuropathies (Nobile-Orazio E. et al., Electroencephalogr Clin Neurophysiol Suppl 1999;50:419); neuromyotonia, acquired neuromyotonia, arthrogryposis multiplex congenita (Vincent A. et al., Ann N Y Acad Sci. 1998 May 13;841:482), Chronic obstructive pulmonary disease (COPD), cardiovascular diseases, cardiovascular autoimmune diseases, atherosclerosis (Matsuura E. et al., Lupus. 1998;7 Suppl 2:S135), myocardial infarction (Vaarala O. Lupus. 1998;7 Suppl 2:S132), thrombosis (Tincani A. et al., Lupus 1998;7 Suppl 2:S107-9), granulomatosis, Wegener's granulomatosis, arteritis, Takayasu's arteritis and Kawasaki syndrome (Praprotnik S. et al., Wien Klin Wochenschr 2000 Aug. 25; 112 (15-16):660); anti-factor VIII autoimmune disease (Lacroix-Desmazes S. et al., Semin Thromb Hemost.2000;26 (2):157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis (Noel L H. Ann Med Interne (Paris). 2000 May; 151 (3):178); antiphospholipid syndrome (Flamholz R. et al., J Clin Apheresis 1999;14 (4):171); heart failure, agonist-like beta-adrenoceptor antibodies in heart failure (Wallukat G. et al., Am J Cardiol. 1999 Jun. 17; 83 (12A):75H), thrombocytopenic purpura (Moccia F. Ann Ital Med Int. 1999 April-June; 14 (2):114); hemolytic anemia, autoimmune hemolytic anemia (Efremov D G. et al., Leuk Lymphoma 1998 January; 28 (3-4):285), gastrointestinal diseases, autoimmune diseases of the gastrointestinal tract, intestinal diseases, chronic inflammatory intestinal disease (Garcia Herola A. et al., Gastroenterol Hepatol. 2000 January; 23 (1):16), celiac disease (Landau Y E. and Shoenfeld Y. Harefuah 2000 Jan. 16; 138 (2):122), Crohn's disease, ulcerative colitis, psoriasis autoimmune diseases of the musculature, myositis, autoimmune myositis, Sjogren's syndrome (Feist E. et al., Int Arch Allergy Immunol 2000 September; 123 (1):92); smooth muscle autoimmune disease (Zauli D. et al., Biomed Pharmacother 1999 June; 53 (5-6):234), hepatic diseases, hepatic autoimmune diseases, autoimmune hepatitis (Manns M P. J Hepatol 2000 August; 33 (2):326) and primary biliary cirrhosis (Strassburg C P. et al., Eur J Gastroenterol Hepatol. 1999 June; 11 (6):595).

According to specific embodiments, the disease is allergy. Specific examples of allergic diseases which may be treated according to the teachings of the present invention include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

According to other specific embodiments, the disease is a graft rejection disease.

Specific examples of graft rejection diseases which may be treated according to the teachings of the present invention include but are not limited to host vs. graft disease, chronic graft rejection, subacute graft rejection, hyperacute graft rejection, acute graft rejection, allograft rejection, xenograft rejection and graft-versus-host disease (GVHD).

Enriching for an MNBC capable of inducing tolerance to an autoimmune cell an allergen or a graft can be effected by any method known in the art (see e.g. Martelli et al. Blood. 2014;123(7):967-973; Raïch-Regué et al. Immunology Letters 161 (2014) 216-221; and Vassalli Journal of Transplantation Volume 2013, Article ID 761429, 17 pages. According to specific embodiments, cells are selected from the group consisting of regulatory DCs, immature DCs and regulatory T cells.

Thus, for example generation of regulatory DC vaccines can induce tolerance. Regulatory DCs (DCreg) induce anergy, promotion of Treg differentiation and induction of T cell death and therefore, thus they can be used for treatment of conditions such as autoimmune diseases, allergy and graft rejection. The DCreg can either be pulsed or non-pulsed with an antigen.

DCregs can be generated by contacting DCs with appropriate cytokines, anti-inflammatory biologicals, or following their genetic modification and propagation in culture. Non-limiting examples of molecules that can induce generation of DCreg include, but are not limited to, IL-10, TGFβ, 1α,25-dihydroxyvitamin D3 (vitD3), hepatocyte growth factor, estrogen, vasoactive intestinal pep-tide (VIP), binding immunoglobulin protein (BiP), thymic stromal lymphopoietin (TSLP), prostaglandin (PG) E2, anti-inflammatory agents (such asacetylsalicylic acid), histamine, adenosine receptor agonists, and immunosuppressive drugs such as corticosteroids, cyclosporine A, rapamycin, deoxyspergualin, tacrolimus (FK506), mycophenolatemofetil (MMF), and BAY-117085, prednisolone or dexamethasone (Dex), rapamycin, BAY-117085 (an irreversible NFkappaB inhibitor). In addition, several genetic manipulations can be used to modulate the maturation of DC to induce DCreg including inducing the expression of different immunomodulatory molecules (such as IL-4, IL-10, TGFβ, CTLA-4, PDL-1, or, in contrast, by inhibiting specific molecules involved in DC activation (i.e.IL-12p35, CD40, or CD86).

Another type of DC that can induce tolerance is an immature DC. Antigen presentation by an immature DC usually results in immune tolerance because of the lack of co-stimulatory molecules. The induction of immune tolerance occurs through various mechanisms including T cell deletion and expansion of Treg.

An example for the generation of T cells that can be used for adoptive T cells transfer for inducing tolerance, the peripheral blood or purified population thereof is enriched ex-vivo for Tregs.

Following enrichment of the desired population of cells these cells can be transplanted to a subject as adoptive therapy or as vaccines for treating the respective disease.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

As used herein, the term “transplanting” refers to the administration of cells, tissues or organs into a subject in need.

The transplantation can be autologous or non-autologous; it can be syngeneic or non-syngeneic: allogeneic or xenogeneic.

As used herein, the term “autologous” means that the donor subject is the recipient subject. Thus, in autologous transplantation the cells have been removed and re-introduced e.g., re-infused to the subject.

According to a specific embodiment, the transplantation is autologous transplantation.

As used herein, the term “non-autologous” means that the donor subject is not the recipient subject.

According to a specific embodiment, the transplantation is non-autologous transplantation.

As used herein, the term “syngeneic” means that the donor subject is essentially genetically identical with the recipient subject. Examples of syngeneic transplantation include transplantation of cells, tissue or organs derived from the subject (also referred to in the art as “autologous”), a clone of the subject, or a homozygotic twin of the subject

As used herein, the term “allogeneic” means that the donor is of the same species as the recipient, but which is substantially non-clonal with the recipient. Typically, outbred, non-zygotic twin mammals of the same species are allogeneic with each other. It will be appreciated that an allogeneic donor may be HLA identical or HLA non-identical with respect to the subject.

As used herein, the term “xenogeneic” means that the donor subject is from a different species relative to the recipient subject.

Thus, according to an aspect of the present invention, there is provided a method of treating cancer in a subject in need thereof, the method comprising:

(a) obtaining cells effective for the treatment of cancer according to the methods of the invention; and

(b) transplanting said cells to a subject,

thereby treating the cancer in the subject.

According to another aspect of the present invention, there is provided a method of treating an infectious disease in a subject in need thereof, the method comprising:

(a) obtaining cells effective for the treatment of an infectious disease in a subject according to the methods of the invention; and

(b) transplanting said cells to a subject,

thereby treating the infectious disease in the subject.

According to another aspect of the present invention, there is provided a method of treating an autoimmune disease, allergy or graft rejection disease in a subject in need thereof, the method comprising:

(a) obtaining cells effective for treatment of an autoimmune disease, allergy or graft rejection disease in a subject according to the methods of the invention; and

(b) transplanting said cells to a subject,

thereby treating the autoimmune disease, allergy or graft rejection disease in the subject.

According to another aspect of the present invention, method of transplanting a graft in a subject in need, the method comprising:

(a) transplanting the graft in the subject;

(b) obtaining cells according to the methods of the invention; and

(c) transplanting said cells to said subject,

thereby transplanting the graft in the subject.

As used herein, the graft may be a cell, a tissue or a whole organ graft. The origin of the graft may be embryonic, fetal, post natal or adult. Thus, the term “graft” refers to a bodily cell (e.g. a single cell or a group of cells) or tissue (e.g. solid tissues or soft tissues, which may be transplanted in full or in part). Exemplary tissues and whole organs which may be transplanted according to the present teachings include, but are not limited to, liver, pancreas, spleen, kidney, heart, lung, skin, intestine and lymphoid/hematopoietic tissues (e.g. lymph node, Peyer's patches, thymus or bone marrow). The cells may be stem cells, progenitors (e.g. immature HSCs) or differentiated cells. Exemplary cells which may be transplanted according to the present teachings include, but are not limited to, hematopoietic stem cells.

As used herein, the tem “hematopoietic stem cell” or “HSC” is used in the broadest sense to refers to stem cells from which blood cells derive, including pluripotent stem cells, lymphoid and myeloid stem cells; as well as to hematopoietic progenitor cells which are the progeny of a pluripotent hematopoietic stem cell which are committed for a particular line of differentiation e.g. erythrocytes, megakaryocytes, monocytes or granulocytes. Typically, HSC are positive for the cell surface marker CD34.

Methods of purifying and transplanting a hematopoietic stem cell graft are well known to one of ordinary skill in the art and are disclosed e.g. in International Patent Application Publication No. WO2008/075369.

Transplanting the graft into the subject may be effected in numerous ways, depending on various parameters, such as, for example, the cell or tissue type; the type, stage or severity of the recipient's disease (e.g. organ failure); the physical or physiological parameters specific to the subject; and/or the desired therapeutic outcome.

Transplanting the graft may be effected into any one of various anatomical locations, depending on the application. The graft may be transplanted into a homotopic anatomical location (a normal anatomical location for the transplant), or into an ectopic anatomical location (an abnormal anatomical location for the transplant). For example, a liver tissue may be transplanted into the liver, the portal vein, the renal capsule, the sub-cutis, the omentum, the spleen, and the intra-abdominal space. Transplantation of a liver into various anatomical locations such as these is commonly practiced in the art to treat diseases amenable to treatment via hepatic transplantation (e.g. hepatic failure). Similarly, transplanting a pancreatic tissue may be effected by transplanting the tissue into the portal vein, the liver, the pancreas, the testicular fat, the sub-cutis, the omentum, an intestinal loop (the subserosa of a U loop of the small intestine) and/or the intra-abdominal space. Transplantation of pancreatic tissue may be used to treat diseases amenable to treatment via pancreatic transplantation (e.g. diabetes). Likewise, transplantation of tissues such as a kidney, a heart, a lung or skin tissue may be carried out into any anatomical location described above for the purpose of treating recipients suffering from, for example, renal failure, heart failure, lung failure or skin damage (e.g., burns).

Optionally, when transplanting a graft into a subject having a defective organ, it may be advantageous to first at least partially remove the failed organ from the subject so as to enable optimal development of the transplant, and structural/functional integration thereof with the anatomy/physiology of the subject.

The order in which the graft and the cells are transplanted in the subject can vary according to the method of treating. Thus, according to a specific embodiment, step (a) is effected prior to step (c).

According to another specific embodiment, step (a) is effected following step (c).

According to yet another specific embodiment, step (a) is effected concomitantly with step (c).

According to specific embodiments, the cells according to this aspect of the present invention cells capable of at least one of:

(i) inducing tolerance to the graft;

(ii) improving post-graft immune reconstitution;

(iii) reducing graft-related mortality; and

(iv) inducing or increasing a GVL effect.

According to specific embodiments, these cells are selected from the group consisting of T cells transduced with a suicide gene, pathogen-specific T cells, leukemia or lymphoma-specific T cells, non-alloreactive T cells, veto cells, TCRγδ⁺ T cells and regulatory T cells.

Methods of enriching for cells endowed with these capabilities are known in the art and disclosed for example in Martelli et al. Blood. 2014;123(7):967-973; Ophir and Reisner Front Immunol. 2012 3:93; Lask et al. Blood. 2013;121(15):3033-3040.

According to specific embodiments, the graft and the cells are syngeneic.

According to a specific embodiment the graft and the cells are autologous. In this case, according to a specific embodiment, the graft is obtained from the donor prior administration of the peptide. According to another specific embodiment, the graft is obtained from the donor following administration of the peptide.

According to specific embodiments, wherein the graft is a hematopoietic stem cell graft and the graft and the cells are autologous, the hematopoietic stem cell graft is obtained prior peptide administration. According to another specific embodiment, wherein the graft is a hematopoietic stem cell graft and the graft and the cells are autologous, the hematopoietic stem cell graft is obtained following peptide administration. In a particular embodiment, wherein the graft is a hematopoietic stem cell graft and the graft and the cells are autologous, the method further comprises purifying said hematopoietic stem cells from said peripheral blood following said collecting.

According specific embodiments, when said cells comprise DCs, said transplanting is in combination with an adjuvant. Suitable non-limiting examples of adjuvants include complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, dinitrophenol, conventional bacterial products (e.g. cholera toxin, heat-labile enterotoxin, attenuated or killed BCG (Bacillus Calmette-Guerin) and Cory bacterium parvum, and BCG derived proteins).

According to specific embodiments, the cells of the invention can be administered to a subject in combination with other established or experimental therapeutic regimen to treat a disease (e.g. cancer) including analgetics, chemotherapeutic agents, radiotherapeutic agents, hormonal therapy and other treatment regimens (e.g., surgery) which are well known in the art.

According to specific embodiments, the subject is further treated with cytotoxic therapies (conditioning) administered before the adoptive cell transfer. This cytotoxic therapy can enhance effectiveness of treatment with the aim (i) to “make space” for the newly administered cells, (ii) to reduce the number of host lymphocytes to prevent allo-reactivity and rejection and (iii) to induce tumor cell apoptosis to reduce the number of tumor cells and increase their immunogenicity.

According to specific embodiments, the cells are transplanted in combination with an anti-cancer immune modulator agent.

As used herein, the term “anti-cancer immune modulator agent” refers to an agent capable of eliciting an immune response (e.g. T cell, NK cell) against a cancerous cell.

According to specific embodiment, the agent is selected from the group consisting of a cancer antigen, a cancer vaccine, an anti-cancer antibody, a cytokine capable of inducing activation and/or proliferation of a T cell and an immune-check point regulator.

The transplantation of the cells of the present invention and the administration of the agent can be effected in the same route or in separate routes.

The transplantation may be prior, following or concomitant with the agent.

According to a specific embodiment, the transplanting the cells of the present invention is effected prior to the treatment with said agent.

According to another specific embodiment, transplanting the cells of the present invention is effected following the treatment with said agent.

According to another specific embodiment, transplanting the cells of the present invention is effected concomitant with the treatment with said agent.

Multiple rounds of transplantation of the cells obtained according to the methods of the present invention and multiple doses of the agent can be administered. Thus, according to specific embodiments, transplanting the cells of the present invention is effected following at least one administration of the agent. According to specific embodiments, administration of the agent is effected following at least one transplantation procedure. According to specific embodiments, transplanting the cells of the present invention is effected in a sequential order with the treatment with the agent.

The peptides, therapeutic agents described hereinabove and/or the cells obtained according to the methods of the invention can be administered to the subject per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism. Optionally, a plurality of different active ingredient may be included in the formulation (i.e., co-formulation) such as an immune-modulating agent, a cytokine that stimulates mobilization of cell to the peripheral blood, chemotherapeutic, radiation agents and the like.

Herein the term “active ingredient” refers to the peptides and/or the cells accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, intradermal, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

According to a specific embodiment, the peptide of the invention or the pharmaceutical composition comprising same is administered subcutaneously.

According to another specific embodiment, the peptide of the invention or the pharmaceutical composition comprising same is administered intravenously.

According to a specific embodiment, the cells of the invention or the pharmaceutical composition comprising same is administered via an intraperitoneal route or via an intrabone route.

According to a specific embodiment, the cells of the invention or the pharmaceutical composition comprising same is administered via an intramuscular, or intradermal route.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Alternative embodiments include depots providing sustained release or prolonged duration of activity of the active ingredient in the subject, as are well known in the art.

Pharmaceutical compositions suitable for use in context of some embodiments of the invention include compositions wherein the active ingredients are contained in an amount effective to achieve the intended purpose. More specifically, according to specific embodiments, a therapeutically effective amount means an amount of active ingredients effective to induce migration of at least one type of mononuclear blood cells (MNBCs) to the peripheral blood of the subject. According to other specific embodiments, a therapeutically effective amount means an amount of active ingredients effective to prevent, alleviate or ameliorate symptoms of a disorder (e.g., cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

According to specific embodiments the peptide of the invention or the pharmaceutical composition comprising same is administered in a dose ranging between 0.1 to10 mg/kg of body weight, between 0.1 to 2 mg/kg of body weight, between 0.1 to 1 mg/kg of body weight, between 0.3 to 10 mg/kg of body weight, between 0.3 to 2 mg/kg of body weight, between 0.3 to 1 mg/kg of body weight or between 0.3 to 0.9 mg/kg of body weight.

According to a specific embodiment, the peptide of the invention or the pharmaceutical composition comprising same is administered in a dose ranging between 0.5-1 mg/kg.

The desired dose can be administered at one time or divided into sub-doses, e.g., 2-4 sub-doses and administered over a period of time, e.g., at appropriate intervals through the day or other appropriate schedule.

According to specific embodiments, the peptide of the invention or the pharmaceutical composition comprising same is administered multiple times e.g. 2-10, over a period of time e.g. for several days to several weeks at appropriate intervals e.g. once a day, twice a week, once a week.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

It is expected that during the life of a patent maturing from this application many relevant immunotherapy strategies will be developed and the scope of the term “enriching at least one type MNBCs effective for the treatment of a disease” is intended to include all such new technologies a priori.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Reagents—BL-8040 (4F-benzoyl-TN14003, SEQ ID NO:1) was manufactured in accordance with cGMP by MSD/N.V. (Organon, Kloosterstraat 6, 5349 AB, Oss, Netherlands).

Leukapheresis—The leukapheresis procedure was commenced approximately 4 hours (±1 hour) post BL-8040 injection. Eighteen (18) Liter apheresis was collected at a Bone Marrow Transplant unit using the Spectra Optia® Apheresis System.

White blood cell count—Blood count assays were performed using Beckman-Coulter analyzers (GENS,750,000).

Flow cytometry—Cells (100 μl) were stained for 30 minutes in 0.1 ml FACS buffer with fluorescence antibodies directed against Human CD3, CD19, CD56, CD4, CD8, CCR7, CD83, lin-cocktail, CD11c, CD45RA, CD25, /Foxp3+, CD16, CD34, CD38 CD105 or matched isotype controls and mouse CD3, and B220 all from eBioscience San Diego, Calif. 92121 USA. Following staining the cells were washed with FACS buffer and analyzed by flow cytometry (5 μl per sample) using the FACS Caliber Flow Cytometer (BD Biosciences). Data was analyzed using software from CellQuest (version 3.3; BD Biosciences). Cells were first gated according to the forward scatter and side scatter parameters to exclude dead cells and the number and percent of each cell population (e.g. HSPCs, T cells, B cells, NK cells, NKT cells and DCs) was analyzed.

### Example 1

### BL-8040 Treatment Induces Mobilization of Hspcs, Mscs, T Cells, B Cells and Dendritic Cells

### Experimental Design:

The first part of the study was a randomized, double-blind, placebo-controlled dose escalation. Each cohort included 8 healthy subjects in a standard 6+2 design (6 subjects receiving active drug and 2 placebo). After obtaining informed consent, BL-8040 was administered subcutaneously (SC) once daily on 2 consecutive days at a dose of 0.5, 0.75 or 1 mg/kg. Frequent peripheral blood (PB) samples were retrieved and tested for the number of white blood cells (WBC), CD34+ hematopoietic stem/progenitor cells (HSPC), B cells, T cells, NK cells, mesenchymal stem cells (MSC), MSC colony forming cells and HPCs colony forming cells.

The second part of the study was an open label test. 8 healthy subjects received a single injection of BL-8040 (1 mg/kg) and 3-4 hours later were subjected to a standard leukapheresis (18 L) procedure. The composition of the collected graft was tested for the number of WBCs per kg, number of CD34+ HSPC per kg and number and type of T cells, B cells, NK cells and dendritic cells (DC) per kg.

### Results:

In the first part of the study BL-8040 was found safe and well tolerated at all doses tested (0.5-1 mg/kg). The primary treatment related adverse effects were mild to moderate transient injection site and systemic reactions. BL-8040 triggered substantial mobilization of WBC to the circulation: The mean WBC count rose from a baseline of 6.3 to 29.7×10⁹/L at 4 hr post BL-8040 administration. In addition, dramatic mobilization of CD34+ HSPC was observed across all doses tested: Mean CD34+ count at baseline was 5.8/μL while four hours post first BL-8040 administration, the count rose to a mean of 8, 37, 31 and 35 cells/μL (placebo, 0.5, 0.75 and 1 mg/kg, respectively). Four hours post second administration of BL-8040, the mean count of CD34+ HSPC further increased to 9, 38, 46 and 58 cells/μL (placebo, 0.5, 0.75 and 1 mg/kg, respectively). Importantly, BL-8040 administration resulted in rapid mobilization of MSC and HPC colony forming cells and substantial mobilization of T, B and NK cells. Long CXCR4 receptor occupancy and long pharmacodynamic effect (≥24 hours post dosing) were also observed.

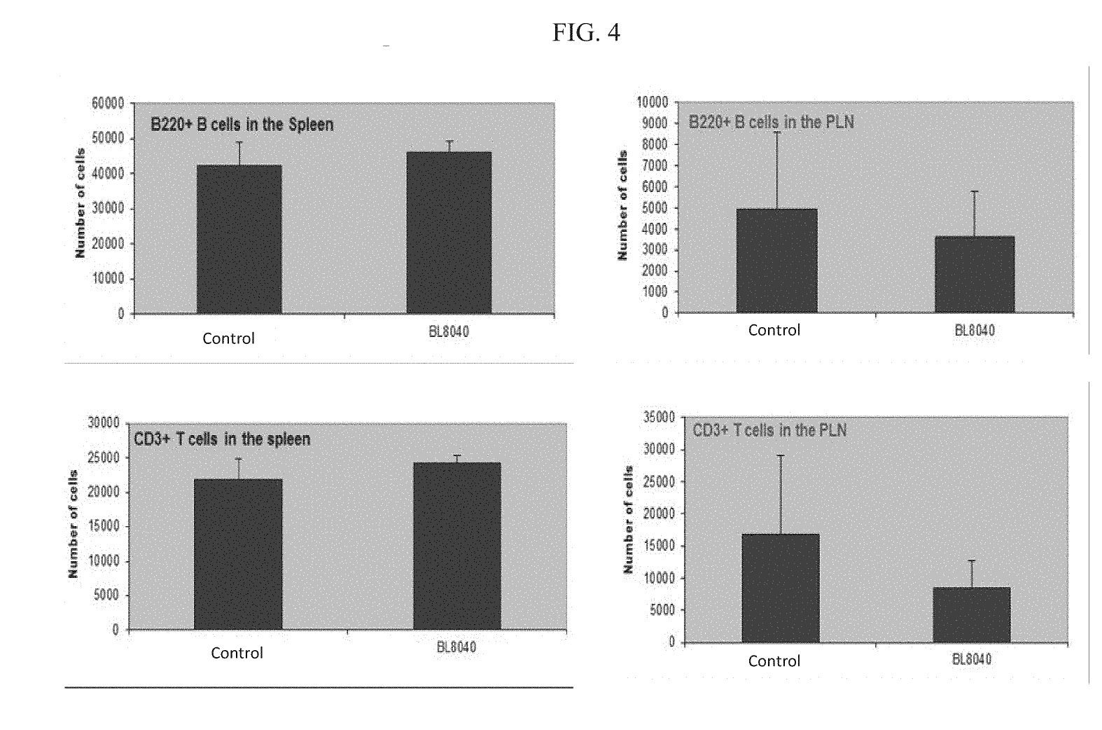

In the second part of the study, 1 mg/kg of BL-8040 stimulated a 5-7 fold increase in the total number of WBC as demonstrated in FIG. 1. In addition, evaluation of the cell composition collected 3.5-8 hours following BL-8040 administration revealed an increase in the number of CD34+ HSPC, T cells, B cells, NK cells, NKT cells and immature DCs (see Table 2 below). The T cell compartment collected in the aphaeresis comprised naïve, memory and effector CD4+ and CD8+ cells (i.e. CD3+/CD4+ CD45RA+/CCR7+ Naïve CD4+ T cells, CD3+/CD4+/CD45RA+/CCR7− Effector CD4+ T cells, CD3+/CD4+/CD45RA−/CCR7− Effector memory CD4+ T cells, CD3+/CD4+/CD45RA-/CCR7+ Central memory CD4+ T cells, CD3+/CD8+/CD45RA+/CCR7+ Naïve CD8+ T cells, CD3+/CD8+/CD45RA+/CCR7− Effector CD8+ T cells, CD3+/CD8+/CD45RA−/CCR7− Effector memory CD8+ T cells and CD3+/CD8+/CD45RA-/CCR7+ Central memory CD8+ T cells, see Table 3 below). Importantly, the number of T cells in the periphery started to decline 48 hours following administration of BL-8040. Experiments in mice have further demonstrated that the T cells are mobilized from the lymph node into the blood therefore may consist of memory and effector CD4+ and CD8+ T cells with the capacity to target tumors (FIG. 4).

In contrast to T cells (FIG. 3), the immature DC (ImDC) were transiently mobilized to the peripheral blood: the number of ImDCs in the periphery declined fast and 24 hours following BL-8040 administration reached the normal levels (see FIG. 2).

Interestingly, BL-8040 administration stimulated a quantitatively distinct mobilization pattern of HSPCs, T cells, B cells and NK cells into the blood circulation as compared to the response induced by the bicyclam CXCR4 inhibitor AMD3100 (Mozobil) or the mobilization agent G-CSF (see Table 4 below).

TABLE 2
Number and percentages of the different cell subsets found in
PB 3.5-8 hours following administration of 1 mg/kg of BL-8040

| | | | | % of |
|---|---|---|---|---|
| Cell subset | Marker | Median (range) | | graft |
| T cells × 10⁸/Kg | CD3+ | 7.32 | (4.0-8.5) | 48.08 |
| T helper cells × | CD3+CD4+ | 3.3 | (1.44-3.9) | 21.7 |
| 10⁸/Kg | | | | |
| Cytotoxic T | CD3+CD8+ | 2.89 | (1.7-4.9) | 22.5 |
| cells × 10⁸/Kg | | | | |
| B cells × 10⁸/Kg | CD19+ | 2.03 | (0.32-2.6) | 13.1 |
| NK cells × 10⁸/Kg | CD3−CD56+ | 0.53 | (0.15-1.55) | 4.76 |
| HSPC × 10⁶/Kg | CD34+ | 11.89 | (5.1-15.0) | 0.78 |
| NKT cells × 10⁶/Kg | CD3+CD56+ | 9.8 | (4.4-47.5) | 1.2 |
| Regulatory T | CD4+CD25+Foxp3+ | 16.1 | (9.7-27.3) | 1.28 |
| cells × 10⁶/Kg | | | | |
| Immature | lin-CD11c+CD83− | 11.6 | (10.3-25.9) | 0.95 |
| DCs × 10⁶/Kg | | | | |

TABLE 3
Percentages of memory and effector CD4+ and CD8+ T cells found
in PB 3.5-8 hours following administration of 1 mg/kg of BL-8040

| Cell subset | Marker | |
|---|---|---|
| | | % of CD4+ |
| CD4+ T cells | | cells |
| Naïve CD4 T cells | CD3+/CD4+ CD45RA+/CCR7+ | 34.61 |
| Effector CD4 T cells | CD3+/CD4+ CD45RA+/CCR7− | 6.24 |
| Effector memory CD4 | CD3+/CD4+ CD45RA−/CCR7− | 21.19 |
| T cells | | |
| Central memory CD4 | CD3+/CD4+ CD45RA−/CCR7+ | 37.99 |
| T cells | | |
| | | % of CD8 |
| CD8+ T cells | | cells |
| Naïve CD8 T cells | CD3+/CD8+ CD45RA+/CCR7+ | 63.89 |
| Effector CD8 T cells | CD3+/CD8+ CD45RA+/CCR7− | 1.09 |
| Effector memory CD8 | CD3+/CD8+ CD45RA−/CCR7− | 1.94 |
| T cells | | |
| Central memory CD8 | CD3+/CD8+ CD45RA−/CCR7+ | 33.1 |
| T cells | | |

TABLE 4
Number of different cell subsets found in PB 3.5-8 hours following
administration of BL-8040, as compared to AMD3100 or G-CSF.

| | | | AMD3100 | Fold | G-CSF from | Fold |
|---|---|---|---|---|---|---|
| | | | from DiPersio | Difference | DiPersio Blood | Difference |
| Cell | | BL-8040 | Blood 2008 | (BL-8040/ | 2008 | (BL-8040/ |
| subset | Marker | Median (range) | Median (range) | AMD3100) | Median (range) | G-CSF) |
| HSPC × | CD34+ | 11.89 (5.1-15.0) | 2.9 (1.2-6.3) | 4.10 | 4.2 (2.5-18) | 2.83 |
| 10⁶/Kg | | | | | | |
| T cells × | CD3+ | 7.32 (4.0-8.5) | 4.6 (1.5-7.8) | 1.59 | 1.3 (1.2-6.8) | 5.63 |
| 10⁸/Kg | | | | | | |
| T helper | CD3+CD4+ | 3.3 (1.44-3.9) | 3.2 (1-5.7) | 1.03 | 1.1 (0.7-3.2) | 3 |
| cells × | | | | | | |
| 10⁸/Kg | | | | | | |
| Cytotoxic | CD3+CD8+ | 2.89 (1.7-4.9) | 1.3 (0.4-3.4) | 2.22 | 0.4 (0.4-3.4) | 7.23 |
| T cells × | | | | | | |
| 10⁸/Kg | | | | | | |
| B cells × | CD19 | 2.03 (0.32-2.6) | 1.0 (0.2-2.4) | 2.00 | ND | |
| 10⁸/Kg | | | | | | |
| NK cells × | CD3−CD56+ | 0.53 (0.15-1.55) | 0.3 (0.1-1.0) | 1.77 | 0.2 (0.2-0.5) | 2.65 |
| 10⁸/Kg | | | | | | |

### Conclusion:

The current data demonstrates that BL-8040 is safe and well tolerated and induces rapid mobilization of HSPC. These results support BL-8040 monotherapy as an effective strategy to shorten the procedure length required to collect sufficient cells for HCT. In addition, treatment with BL-8040 yielded a potent hematopoietic graft with unique cell composition which may also serve as a novel approach to collect HPCs as well as T cells, B cells, NK cells, NKT cells and ImDC for immunotherapy.

### Example 2

### Use Of Bl-8040 for Obtaining Dendritic Cells that Can be Used to Produce an Anti-tumor Vaccine

A critical step in vaccination is the efficient presentation of cancer antigens to T cells. As DCs are the most efficient antigen presenting cells, exploiting their diversity, in terms of subsets as well as plasticity, is likely to yield improved therapeutic anti-cancer vaccines. DCs can be expanded in vitro and challenged with a wide variety of cancer-specific antigens. Following this presentation under suitable culture conditions, such as adjuvants, cytokines and co-stimulatory molecules, the DC will capture, process and present the cancer antigen.

Thus, according to an embodiment of the present invention BL-8040 is used to induce migration of DCs and subsequent production of an anti-tumor DC vaccine.

Experimental Procedures

BL-8040 is administered into cancer patients at doses of 0.5-1 mg/kg. DCs are isolated from the peripheral blood of the subject by leukapheresis 4-8 hours following administration.

The immature DCs are purified using Lin-, CD11c+CD8+ FACS sorting or by Milteny column that binds cells that express CD11c, or by columns that select out Lin-cells.

Following purification, the isolated DCs are expanded using tumor antigens such as proteins and peptides known to be overexpressed by the tumors (e.g., MART-1, NY-ESO-1 and Mage-3), neoantigens isolated from the patient's tumor or protein extracts from the tumors. Alternatively, stimulation of DCs is performed by transfection of mRNA coding for cancer antigens.

Co-stirnulation of DCs in culture can be effected using CD40, TLR and/or IFN-γ.

Cytokines such as (TNF)-α, IL-1β, IL-6, and prostaglandin E2 can be used to induce maturation of DCs in culture following capture of the antigen.

The resulting DCs are used as vaccines with or without an adjuvant to present these antigens to T cells. As combination immunotherapies involving DCs and immunomodulatory antibodies may hold promise, the resulting DC vaccine may be administered in combination with antibodies such as anti-CTLA4, anti-PD-1, anti-PDL-1, CD40 agonist, 4-1BB agonist, GITR agonist and OX40 agonist.

### Example 3

### Use pf BL-8040 for Obtaining T Cells that Can be Used for Adoptive Transfer Therapy

Adoptive cell transfer for cancer treatment involves the selection of lymphocytes with anti-tumor activity, their expansion/activation ex-vivo, and their infusion into the patient, often in the context of lympho-depleting regimens to minimize endogenous immune suppression.

### Example 3A

### Use of BL-8040 for Autologous Cancer Therapy

Experimental Procedures

BL-8040 is administered into cancer patients at doses of 0.5-1 mg/kg. The administration of BL-8040 induces mobilization of T cells and DCs as described in Example 1 above. As the mobilization of these subsets of cells is rapid the CD3+ T cells and optionally DCs are collected by apheresis or by collecting blood 3-4 hours following the administration of BL-8040. The number of T cells in the periphery start to decline only 48 hours following administration of BL-8040 therefore leaving time for additional collection if needed.

CD3+, CD4+, CD8+ or T cells or tumor-associated lymphocytes (TALs) selected for T-cell receptor (TCR) specificity (Cancer Immunol Immunother. 2009;58:553-66.), are then purified by FACS or magnetic beads separation. The cells are either frozen or used immediately for ex-vivo expansion by incubation with mature autologous DCs preloaded with tumor antigens or transfected with mRNA coding for cancer antigens. The DCs used are purified according to the teachings in Example 2 above or using any method well known in the art such as DCs derived from monocytes.

The expanded T cells and optionally the DCs are then transplanted to the patient. Patients can be further treated with T cells activators such as IL-2 and IFN-α and/or with immunomodulatory antibodies such as anti-CTLA4, anti-PD-1, anti-PDL-1, CD40 agonist, 4-1BB agonist, GITR agonist and OX40 agonist.

### Example 3B

### Use of BL-8040 for Hematologic Malignancies

Experimental Procedures

BL-8040 is administered into hematopoietic stem cell donors at doses of 0.5-1 mg/kg. The administration of BL-8040 induces mobilization of T cells and DCs as described in Example 1 above. As the mobilization of these subsets of cells is rapid the CD3+ T cells and optionally DCs are collected by apheresis or by collecting blood 3-4 hours following the administration of BL-8040. The number of T cells in the periphery starts to decline only 48 hours following administration of BL-8040 therefore leaving time for additional collection if needed.

In this context, the donor of the T cells and DCs is the same donor of the hematopoietic stem cell transplant. The stem cells transplant can be obtained prior to BL-8040 or following BL-8040 administration e.g. together with harvesting of the T cells and DCs.

CD3+, CD4+, CD8+ or T cells or tumor-associated lymphocytes (TALs) selected for T-cell receptor (TCR) specificity [Cancer Immunol Immunother. (2009) 58: 553-66] are purified by FACS or magnetic beads separation. The cells are either frozen or used immediately for ex-vivo expansion by incubation with mature donor DCs preloaded with tumor antigens or transfected with mRNA coding for cancer antigens. The DCs used are purified according to the teachings of Example 2 above or using any method well known in the art such as DCs derived from monocytes. The expanded T cells and optionally the DCs are then transplanted to the allogeneic cancer patient. Patients can be further treated with T cells activators such as IL-2 and IFNα and/or with immunomodulatory antibodies such as anti-CTLA4, anti-PD-1, anti-PDL-1, CD40 agonist, 4-1BB agonist, GITR agonist and OX40 agonist.

### Example 3C

### Use of BL-8040 for Preparation of CAR T Cells

One approach for expanding and processing T cells in a way that allows specific targeting to tumor antigens and cell surface molecules is to create tumor-reactive T-cell populations from PBL by retrovirally transducing them with chimeric antigen receptors (CAR) to tumor-associated antigens or natural T-cell receptors against antigens presented in the context of MHC.[Journal of clinical oncology: official journal of the American Society of Clinical Oncology. (2011); 29(7):917-924].

Experimental Procedures

BL-8040 is administered into cancer patients at doses of 0.5-1 mg/kg. The administration of BL-8040 induces mobilization of T cells and DCs as described in Example 1 above. As the mobilization of these subsets of cells is rapid the CD3+ T cells are collected by apheresis or by collecting blood 3-4 hours following the administration of BL-8040. The number of T cells in the periphery start to decline only 48 hours following administration of BL-8040 therefore leaving time for additional collection if needed.

CD3+, CD4+, CD8+ or T cells or tumor-associated lymphocytes (TALs) selected for T-cell receptor (TCR) specificity [Cancer Immunol Immunother. (2009) 58: 553-66], are then purified by FACS or magnetic beads separation. Cell are either frozen or used immediately for preparation of CAR T cells. The expanded CAR T cells are then transplanted to the cancer patient. Patients can be further treated with T cells activators such as IL-2 and IFNα and/or with immunomodulatory antibodies such as anti-CTLA4, anti-PD-1, anti-PDL-1, CD40 agonist, 4-1BB agonist, GITR agonist and OX40 agonist.

### Example 4

### The Generation of Immune Graft for Adoptive Transfer Therapy

BL8040 is injected into a tumor-bearing subject and cells collected from the blood 2-24 hr later. The mononuclear cells are purified on Ficoll and incubated on irradiated tumor cells or tumor antigens in the presence of cytokines such as IL-2 and IL-15 and GM-CSF and antibodies to immune checkpoints such as anti PD-1 for 1-30 days. Expended cells are collected and administrated into patients with and without cytokine(s) of other immunomodulators such as anti PD-1.

Experimental Procedures

Mice

Six-to 8-week-old female or male C57BL/6 were purchased from Harlan laboratories. Mice were maintained and all animal experiments were conducted according to the protocols approved by the institutional animal care of the Hebrew university.

Cell Lines

The B 16F10 melanoma cell line was purchased from the ATCC® CRL. 6475™).

The liver metastasis cell line, Livmet, was derived from KrasG12D/+ transgenic mice (Tuveson et al. 2006 Cancer Res. 66(1):242-7), which developed pancreatic ductal adenocarcinoma (PDA) and liver metastasis. Cells were cultured in DMEM medium supplemented with 10% heat-inactivated fetal calf serum (FCS), 1 mmol/L L-glutamine, sodium pyruvate, 100 U/mL penicillin, and 0.01 mg/mL streptomycin (Biological Industries) in a humidified atmosphere of 5% CO₂ at 37 ° C.

Mice Model

Female C57BL/6 mice were inoculated subcutaneously at the right flank with 1× 10⁶ B16F10 cells in 0.1 mL PBS. When tumors reached 90 to 150 mm² in size (after two weeks), mice were treated with BL-8040, according to the below regimen.

1.5×10⁶ Livmet cells/10 μl PBS and 10 μl Matrigel (BD Biosciences) were injected into the pancreas of male C57BL/6 mice. Mice were treated with BL8040, 3 weeks after the injection.

BL8040 (400 μg/mouse dissolved in phosphate-buffered saline) was administered once by subcutaneous injection and 2 h later the blood was collected into tube with heparin.

In Vitro Stimulation and Expansion of PBMC

Following mobilization with BL8040, peripheral blood cells were collected from naïve or tumor-bearing mice (pancreatic cancer and melanoma). Mononuclear cells were isolated by standard Ficoll density gradient centrifugation and resuspended in RPMI medium supplemented with 10% heat-inactivated fetal calf serum (FCS), 1 mmol/L L-glutamine, sodium pyruvate, 100 U/mL penicillin, and 0.01 mg/mL streptomycin (Biological Industries). PBMCs were stimulated at 1×10⁶ cells/well with 1 μg/ml anti-CD3 (clone:145-2C11, eBioscience), 5 μg/ml anti-PD-1 (clone:RMP1-14, Biolegend) and 1000IU/m1 Human-IL-2 (R&D systems) in the presence of a 1:10 ratio of 55Gy irradiated cells (B16F10 or Livmet). The stimulation procedure was performed at 37 ° C. and 5% CO₂ in a 24-well plate. On Day 10 cells were analyzed by FACS.

Flow Cytometry Analysis

Fluorescent-activated cell sorting analysis was performed on whole blood (day 0) and expanded cells (day 10).

Whole blood was treated with red blood cell lysis solution (0 155 M NH₄Cl, 0.01 M KHCO₃, 0.01 mmol/l EDTA, pH 7.4) in order to lyse the erythrocytes. Isolated cells were washed with PBS, re-suspended with 0.1 ml of PBS and stained with fluorescent antibodies directed against specific surface markers.

Cells were incubated with mobs: CD8-PE, CD4-APC, CD3-FITC, CD25-PE-Cy7, CD69-PerCP, CD44-PE-Cy7, CD137-PE-Cy7, CD279-FITC, CD62L-FITC and CD223-FITC (eBioscience).

Results

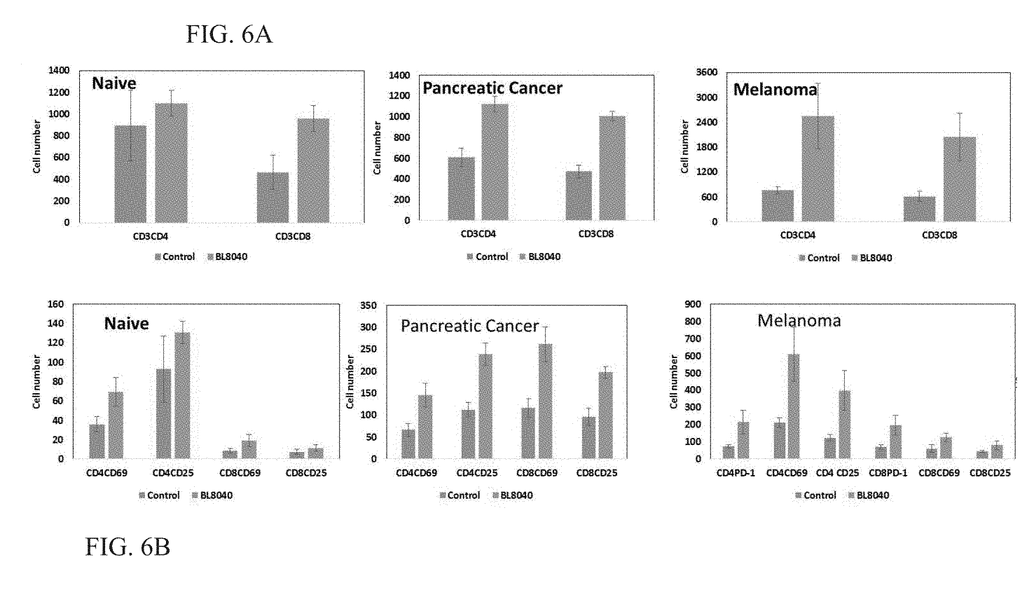

Naïve mice or mice bearing pancreatic or melanoma cancer were injected with BL8040 (400 μg/mouse SC). As shown in FIG. 6A an increased mobilization of both CD4 and CD8 cells is evident in tumor bearing mice compared to control non injected mice or control naïve nice which were either injected with BL8040 or not. FIG. 6B shows that cells collected from cancerous mice contained more CD69+ CD25+ activated T cells as compared to naïve mice.

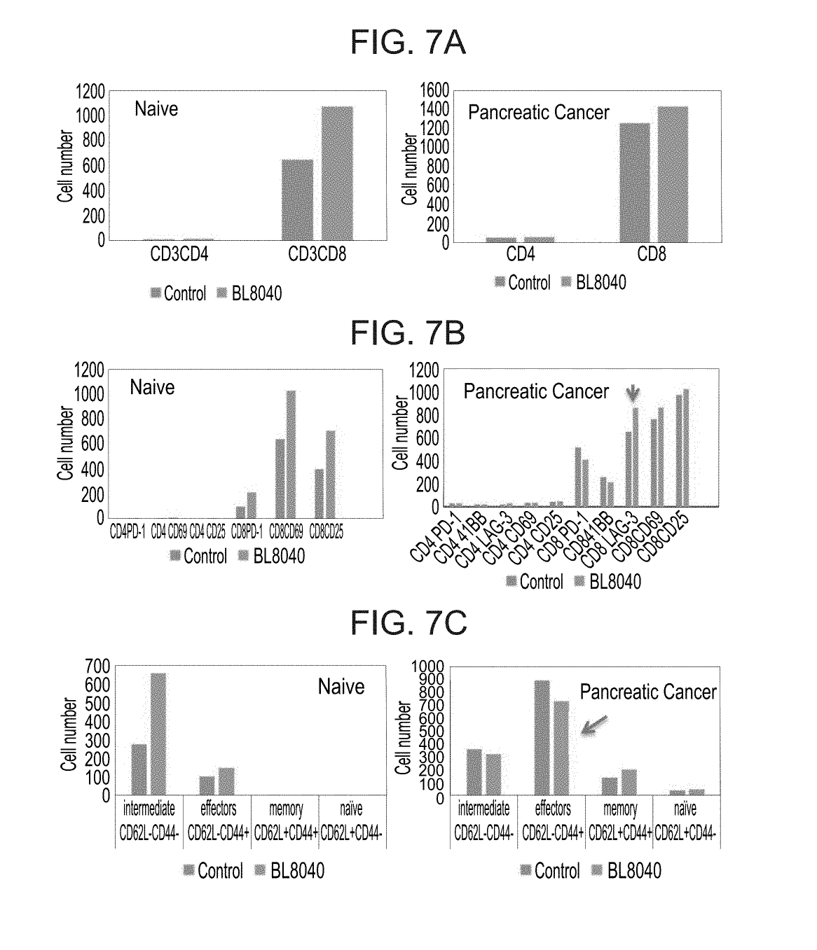

Equal number of PBMC cells that were collected following mobilization or from non mobilized cells were seeded on irradiated pancreatic in the presence of IL-2 and anti PD1 for 11 days. FIG. 7A shows that in all cultures CD8+ cells proliferate selectively in the cultures. FIG. 7 B shows that both cells collected from naïve mice and mice with pancreatic cancer were activated CD69+ CD25+ cells. Cells collected from mice with pancreatic tumors showed a prominent LAGS expression, being a TILL marker. FIG. 7C shows that Cells collected from pancreatic tumors were mainly effector T cells (CD44+), whereas cells collected from naïve mice were mainly intermediate T cells (CD44−).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of obtaining dendritic cells (DCs) from a subject, the method comprising:
(a) administering to the subject an effective amount of a peptide having an amino acid sequence as set forth in SEQ ID NO:1 or an analog or derivative thereof; and
(b) collecting peripheral blood of said subject 4-8 hours following said administering;
thereby obtaining the DCs from the subject.

2. A method of obtaining at least one type of mononuclear blood cells (MNBCs) selected from the group consisting of T cells, B cells, NK cells and NKT cells from a subject, the method comprising:
(a) administering to the subject an effective amount of a peptide having an amino acid sequence as set forth in SEQ ID NO:1 or an analog or derivative thereof;
(b) collecting peripheral blood of said subject 4-48 hours following said administering; and optionally
(c) repeating step (b) at least once no later than 48 hours following said administering;
thereby obtaining the at least one type of MNBCs from the subject.

3. The method of claim 2, wherein said MNBCs comprise T cells.

4. The method of claim 2, further comprising purifying said MNBCs from said peripheral blood following said collecting.

5. A method of obtaining cells effective for the treatment of cancer, the method comprising:
(a) administering to a subject an effective amount of a peptide having an amino acid sequence as set forth in SEQ ID NO:1 or an analog or derivative thereof;
(b) collecting peripheral blood of said subject; and
(c) enriching from said peripheral blood at least one type of mononuclear blood cells (MNBCs) capable of eliciting an immune response against a cancerous cell,
thereby obtaining the cells effective for the treatment of cancer.

6. The method of claim 5, wherein said enriching comprises purifying at least one type of MNBCs selected from the group consisting of dendritic cells (DCs), T cells, B cells, NK cells and NKT cells from said peripheral blood following said collecting.

7. The method of claim 5, wherein said cells are selected from the group consisting of dendritic cells (DCs), T cells, B cells, NK cells and NKT cells.

8. The method of claim 5, wherein said cells comprise dendritic cells (DCs).

9. The method of claim 8, wherein said DCs comprise immature DCs.

10. The method of claim 5, wherein when said cells comprise immature DCs the method comprises inducing maturation of said immature DCs or T cells.

11. (canceled)

12. The method of claim 5, wherein said enriching is effected by a method selected from the group consisting of:
(i) selecting anti-cancer reactive cells;
(ii) activating anti-cancer reactive cells;
(iii) expanding anti-cancer reactive cells;
(iv) promoting presentation of a cancer antigen; and
(v) promoting presentation of an anti-cancer receptor.

13. The method of claim 12, wherein said enriching comprises contacting said peripheral blood or a purified population of cells thereof with a cancer antigen selected from the group consisting of a cancer antigenic peptide or polypeptide, a cancer cell lysate, a cancerous cell and a DC presenting a cancer antigen.

14. The method of claim 12, wherein said activating or expanding comprises contacting said peripheral blood or a purified population of cells thereof with a cytokine capable of inducing activation and/or proliferation of a T cell.

15. The method of claim 12, wherein said activating comprises contacting said peripheral blood or a purified population of cells thereof with a co-stimulatory molecule.

16. The method of claim 15, wherein said co-stimulatory molecule is selected form the group consisting of an immune-check point regulator, LPS and TLR ligands.

17. The method of claim 16, wherein said immune-check point regulator is selected from the group consisting of anti-CTLA4, anti-PD-1 and CD40 agonist.

18. The method of claim 12, wherein when said cells comprise T cells, said promoting presentation of an anti-cancer receptor comprises transducing with a T cell receptor (TCR) or a chimeric antigen receptor (CAR).

19. The method of claim 12, wherein when said cells comprise DCs said promoting presentation of a cancer antigen comprises transfecting with an mRNA coding for a cancer antigen.

20. A method of treating cancer in a subject in need thereof, the method comprising:
(a) obtaining cells effective for the treatment of cancer according to the method of claim 5; and
(b) transplanting said cells to a subject, thereby treating the cancer in the subject.

21. The method of claim 20, wherein when said cells comprise DCs, said transplanting is in combination with an adjuvant.

22. The method of claim 20, wherein said transplanting is in combination with an anti-cancer immune modulator agent.

23. The method of claim 22, wherein said transplanting is effected prior to the treatment with said agent.

24. The method of claim 22, wherein said transplanting is effected concomitant with the treatment with said agent.

25. The method of claim 22, wherein said transplanting is effected following the treatment with said agent.

26. The method of claim 22, wherein said agent is selected from the group consisting of a cancer antigen, a cancer vaccine, an anti-cancer antibody, a cytokine capable of inducing activation and/or proliferation of a T cell and an immune-check point regulator.

27. The method of claim 14, wherein said cytokine is selected from the group consisting of IL-2, IFNα and IL-12.

28. (canceled)

29. A method of obtaining cells effective for the treatment of an infectious disease, the method comprising:
(a) administering to a subject an effective amount of a peptide having an amino acid sequence as set forth in SEQ ID NO:1 or an analog or derivative thereof;
(b) collecting peripheral blood of said subject; and
(c) obtaining from said peripheral blood at least one type of mononuclear blood cells (MNBCs) capable of eliciting an immune response against a pathogen,
thereby obtaining the cells effective for the treatment of the infectious disease.

30. The method of claim 29, wherein said cells are selected from the group consisting of memory T cells, pathogen-specific T cells and DCs presenting a pathogenic antigen.

31. A method of treating an infectious disease in a subject in need thereof, the method comprising:
(a) obtaining cells effective for the treatment of an infectious disease in a subject according to the method of claim 29; and
(b) transplanting said cells to a subject,
thereby treating the infectious disease in the subject.

32. A method of obtaining cells effective for treatment of an autoimmune disease, allergy or graft rejection disease, the method comprising:
(a) administering to a subject an effective amount of a peptide having an amino acid sequence as set forth in SEQ ID NO:1 or an analog or derivative thereof;
(b) collecting peripheral blood from said subject;
(c) obtaining from said peripheral blood at least one type of mononuclear blood cells (MNBCs) capable of inducing tolerance to an autoimmune cell an allergen or a graft;
thereby obtaining the cells effective for treatment of the autoimmune disease, allergy or graft rejection disease.

33. The method of claim 32, wherein said cells are selected from the group consisting of regulatory DCs, immature DCs and regulatory T cells.

34. A method of treating an autoimmune disease, allergy or graft rejection disease in a subject in need thereof, the method comprising:
(a) obtaining cells effective for treatment of an autoimmune disease, allergy or graft rejection disease in a subject according to the method of claim 32; and
(b) transplanting said cells to a subject,
thereby treating the autoimmune disease, allergy or graft rejection disease in the subject.

35. A method of transplanting a graft in a subject in need, the method comprising:
(a) transplanting the graft in the subject;
(b) obtaining cells according to the method of claim 1; and
(c) transplanting said cells to said subject,
thereby transplanting the graft in the subject.

36-50. (canceled)

51. The method of claim 1, wherein said peptide is as set forth in SEQ ID NO: 1.

52. The method of claim 1, wherein said cells do not comprise CD34+ hematopoietic stem/progenitor cells.


SEQUENCE LISTING (72 sequences)

SEQ ID NO: 1 - protein, 14 aa, Artificial
  synthetic peptide
  MOD_RES (1)..(1): 4-fluorobenzoyl-arginine
  MOD_RES (3)..(3): 3-((2-naphthyl) alanine
  MOD_RES (6)..(6): citrulline
  MOD_RES (8)..(8): D-lysine
  MOD_RES (12)..(12): citrulline
  MOD_RES (14)..(14): C′ AMIDATED
XRXCYXKXPYRXCR

SEQ ID NO: 2 - protein, 14 aa, Artificial
  SYNTHETIC PEPTIDE
  MOD_RES (1)..(1): N′ ACETYLATION
  MOD_RES (3)..(3): 3-((2-naphthyl) alanine
  MOD_RES (6)..(6): citrulline
  MOD_RES (8)..(8): D-lysine
  MOD_RES (12)..(12): citrulline
RRXCYXKXPYRXCR

SEQ ID NO: 3 - protein, 14 aa, Artificial
  SYNTHETIC PEPTIDE
  MOD_RES (1)..(1): N′ ACETYLATION
  MOD_RES (3)..(3): 3-((2-naphthyl) alanine
  MOD_RES (8)..(8): D-citrulline
  MOD_RES (12)..(12): citrulline
RRXCYRKXPYRXCR

SEQ ID NO: 4 - protein, 14 aa, Artificial
  synthetic peptide
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-citrulline
  MOD_RES (12)..(12): citrulline
RRXCYXKXPYRXCR

SEQ ID NO: 5 - protein, 14 aa, Artificial
  synthetic peptide
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-lysine
  MOD_RES (11)..(12): citrulline
RRXCYXKXPYXXCR

SEQ ID NO: 6 - protein, 14 aa, Artificial
  synthetic peptide
  MOD_RES (1)..(1): N′ acetylated citrulline
  MOD_RES (3)..(3): 3-((2-naphthyl) alanine
  MOD_RES (6)..(6): citrulline
  MOD_RES (8)..(8): D-lysine
  MOD_RES (12)..(12): citrulline
XRXCYXKXPYRXCR

SEQ ID NO: 7 - protein, 14 aa, Artificial
  synthetic peptide
  MOD_RES (1)..(1): N′ acetylated citrulline
  MOD_RES (3)..(3): 3-((2-naphthyl) alanine
  MOD_RES (8)..(8): D-citrulline
  MOD_RES (12)..(12): citrulline
XRXCYRKXPYRXCR

SEQ ID NO: 8 - protein, 14 aa, Artificial
  synthetic peptide
  MOD_RES (1)..(1): N′ ACETYLATED
  MOD_RES (3)..(3): 3-((2-naphthyl) alanine
  MOD_RES (8)..(8): D-citrulline
  MOD_RES (11)..(12): citrulline
RRXCYRKXPYXXCR

SEQ ID NO: 9 - protein, 14 aa, Artificial
  synthetic peptide
  MOD_RES (1)..(1): N′ ACETYLATED citrulline
  MOD_RES (3)..(3): 3-((2-naphthyl) alanine
  MOD_RES (8)..(8): D-lysine
  MOD_RES (11)..(12): citrulline
XRXCYRKXPYXXCR

SEQ ID NO: 10 - protein, 14 aa, Artificial
  synthetic peptide
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-citrulline
  MOD_RES (12)..(12): citrulline
  MOD_RES (14)..(14): C′ AMIDATED
RRXCYXKXPYRXCR

SEQ ID NO: 11 - protein, 14 aa, Artificial
  synthetic peptide
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-lysine
  MOD_RES (11)..(12): citrulline
  MOD_RES (14)..(14): C′ AMIDATED
RRXCYXKXPYXXCR

SEQ ID NO: 12 - protein, 14 aa, Artificial
  synthetic peptide
  MOD_RES (1)..(1): N′ ACETYLATED citrulline
  MOD_RES (3)..(3): 3-((2-naphthyl) alanine
  MOD_RES (6)..(6): citrulline
  MOD_RES (8)..(8): D-lysine
  MOD_RES (12)..(12): citrulline
  MOD_RES (14)..(14): AMIDATED
XRXCYXKXPYRXCR

SEQ ID NO: 13 - protein, 14 aa, Artificial
  synthetic peptide
  MOD_RES (1)..(1): N′ ACETYLATED citrulline
  MOD_RES (3)..(3): 3-((2-naphthyl) alanine
  MOD_RES (8)..(8): D-citrulline
  MOD_RES (12)..(12): citrulline
  MOD_RES (14)..(14): C′ AMIDATED
XRXCYRKXPYRXCR

SEQ ID NO: 14 - protein, 14 aa, Artificial
  synthetic peptide
  MOD_RES (1)..(1): N′ ACETYLATED
  MOD_RES (3)..(3): 3-((2-naphthyl) alanine
  MOD_RES (8)..(8): D-citrulline
  MOD_RES (11)..(12): citrulline
  MOD_RES (14)..(14): C′ AMIDATED
RRXCYRKXPYXXCR

SEQ ID NO: 15 - protein, 14 aa, Artificial
  synthetic peptide
  MOD_RES (1)..(1): N′ ACETYLATED citrulline
  MOD_RES (3)..(3): 3-((2-naphthyl) alanine
  MOD_RES (8)..(8): D-lysine
  MOD_RES (11)..(12): citrulline
  MOD_RES (14)..(14): C′ AMIDATED
XRXCYRKXPYXXCR

SEQ ID NO: 16 - protein, 14 aa, Artificial
  synthetic peptide
  MOD_RES (1)..(1): D-glutamic acid
  MOD_RES (3)..(3): 3-((2-naphthyl) alanine
  MOD_RES (8)..(8): D-lysine
  MOD_RES (12)..(12): citrulline
XRXCYRKXPYRXCR

SEQ ID NO: 17 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
REXCYRKXPYRXCR

SEQ ID NO: 18 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYEKXPYRXCR

SEQ ID NO: 19 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYREXPYRXCR

SEQ ID NO: 20 - protein, 14 aa, Artificial
  synthetic peptide
  MOD_RES (3)..(3): 3-((2-naphthyl) alanine
  MOD_RES (8)..(8): D-glutamic acid
  MOD_RES (12)..(12): citrulline
RRXCYRKXPYRXCR

SEQ ID NO: 21 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYRKXPYEXCR

SEQ ID NO: 22 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYRKXPYRXCE

SEQ ID NO: 23 - protein, 14 aa, Artificial
  synthetic peptide
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): C′ amidated
RRXCYXKXPYRXCR

SEQ ID NO: 24 - protein, 14 aa, Artificial
  synthetic peptide
  MOD_RES (3)..(3): 3-((2-naphthyl) alanine
  MOD_RES (6)..(6): D-glutamic acid
  MOD_RES (8)..(8): D-citrulline
  MOD_RES (12)..(12): citrulline
  MOD_RES (14)..(14): c′ amidated
RRXCYXKXPYRXCR

SEQ ID NO: 25 - protein, 14 aa, Artificial
  synthetic peptide
  MOD_RES (3)..(3): 3-((2-naphthyl) alanine
  MOD_RES (6)..(6): D-glutamic acid
  MOD_RES (8)..(8): D-glutamic acid
  MOD_RES (12)..(12): citrulline
  MOD_RES (14)..(14): c′ amidated
RRXCYXKXPYRXCR

SEQ ID NO: 26 - protein, 14 aa, Artificial
  synthetic peptide
  MOD_RES (1)..(1): D-glutamic acid
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
XRXCYXKXPYRXCR

SEQ ID NO: 27 - protein, 14 aa, Artificial
  synthetic peptide
  MOD_RES (3)..(3): 3-((2-naphthyl) alanine
  MOD_RES (6)..(6): citrulline
  MOD_RES (8)..(8): D-glutamic acid
  MOD_RES (10)..(10): D-glutamic acid
  MOD_RES (12)..(12): citrulline
  MOD_RES (14)..(14): c′ amidated
RRXCYXKXPXRXCR

SEQ ID NO: 28 - protein, 14 aa, Artificial
  synthetic peptide
  MOD_RES (3)..(3): 3-((2-naphthyl) alanine
  MOD_RES (6)..(6): citrulline
  MOD_RES (8)..(8): D-glutamic acid
  MOD_RES (12)..(12): D-glutamic acid
  MOD_RES (14)..(14): c′ amidated
RRXCYXKXPYRXCR

SEQ ID NO: 29 - protein, 14 aa, Artificial
  synthetic peptide
  MOD_RES (1)..(1): N′ ACETYLATED D-glutamic acid
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
XRXCYXKXPYRXCR

SEQ ID NO: 30 - protein, 14 aa, Artificial
  synthetic peptide
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-glutamic acid
  MOD_RES (10)..(10): D-glutamic acid
  MOD_RES (12)..(12): citrulline
  MOD_RES (14)..(14): c′ amidated
RRXCYXKXPXRXCR

SEQ ID NO: 31 - protein, 14 aa, Artificial
  synthetic peptide
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-glutamic acid
  MOD_RES (12)..(12): D-glutamic acid
  MOD_RES (14)..(14): c′ amidated
RRXCYXKXPYRXCR

SEQ ID NO: 32 - protein, 14 aa, Artificial
  synthetic peptide
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
RRXCYXKXPYRXCR

SEQ ID NO: 33 - protein, 14 aa, Artificial
  synthetic peptide
  MOD_RES (1)..(1): Guanyl-arginine
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
XRXCYXKXPYRXCR

SEQ ID NO: 34 - protein, 14 aa, Artificial
  synthetic peptide
  MOD_RES (1)..(1): Tetramethylguanyl-arginine
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
XRXCYXKXPYRXCR

SEQ ID NO: 35 - protein, 13 aa, Artificial
  synthetic peptide
  MOD_RES (1)..(1): Tetramethylguanyl-arginine
  MOD_RES (2)..(2): 3-((2-naphthyl) alanine
  MOD_RES (5)..(5): citrulline
  MOD_RES (7)..(7): D-glutamic acid
  MOD_RES (11)..(11): citrulline
  MOD_RES (13)..(13): c′ amidated
XXCYXKXPYRXCR

SEQ ID NO: 36 - protein, 14 aa, Artificial
  synthetic peptide
  MOD_RES (1)..(1): 4-fluorobenzoyl-arginine
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): amidated
XRXCYXKXPYRXCR

SEQ ID NO: 37 - protein, 14 aa, Artificial
  synthetic peptide
  MOD_RES (1)..(1): 2-fluorobenzoyl-arginine
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
XRXCYXKXPYRXCR

SEQ ID NO: 38 - protein, 13 aa, Artificial
  synthetic peptide
  MOD_RES (1)..(1): 5-aminopentanoyl-arginine
  MOD_RES (2)..(2): 3-((2-naphthyl) alanine
  MOD_RES (5)..(5): citrulline
  MOD_RES (7)..(7): D-glutamic acid
  MOD_RES (11)..(11): citrulline
  MOD_RES (13)..(13): c′ amidated
XXCYXKXPYRXCR

SEQ ID NO: 39 - protein, 14 aa, Artificial
  synthetic peptide
  MOD_RES (1)..(1): 2-desamino-arginyl
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
XRXCYXKXPYRXCR

SEQ ID NO: 40 - protein, 13 aa, Artificial
  synthetic peptide
  MOD_RES (1)..(1): Guanyl-arginine
  MOD_RES (2)..(2): 3-((2-naphthyl) alanine
  MOD_RES (5)..(5): citrulline
  MOD_RES (7)..(7): D-glutamic acid
  MOD_RES (11)..(11): citrulline
  MOD_RES (13)..(13): c′ amidated
XXCYXKXPYRXCR

SEQ ID NO: 41 - protein, 13 aa, Artificial
  synthetic peptide
  MOD_RES (1)..(1): Succinyl-arginine
  MOD_RES (2)..(2): 3-((2-naphthyl) alanine
  MOD_RES (5)..(5): citrulline
  MOD_RES (7)..(7): D-glutamic acid
  MOD_RES (11)..(11): citrulline
  MOD_RES (13)..(13): c′ amidated
XXCYXKXPYRXCR

SEQ ID NO: 42 - protein, 13 aa, Artificial
  synthetic peptide
  MOD_RES (1)..(1): Glutaryl-arginine
  MOD_RES (2)..(2): 3-((2-naphthyl) alanine
  MOD_RES (5)..(5): citrulline
  MOD_RES (7)..(7): D-glutamic acid
  MOD_RES (11)..(11): citrulline
  MOD_RES (13)..(13): c′ amidated
XXCYXKXPYRXCR

SEQ ID NO: 43 - protein, 14 aa, Artificial
  synthetic peptide
  MISC_FEATURE (1)..(1): desaminoTMG-APA (formula IV in the specification)
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
XRXCYXKXPYRXCR

SEQ ID NO: 44 - protein, 14 aa, Artificial
  synthetic peptide
  MISC_FEATURE (1)..(1): R-CH2 - formula (V) in the specification
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
XRXCYXKXPYRXCR

SEQ ID NO: 45 - protein, 13 aa, Artificial
  synthetic peptide
  MOD_RES (2)..(2): 3-((2-naphthyl) alanine
  MOD_RES (5)..(5): citrulline
  MOD_RES (7)..(7): D-glutamic acid
  MOD_RES (11)..(11): citrulline
  MOD_RES (13)..(13): c′ amidated
RXCYXKXPYRXCR

SEQ ID NO: 46 - protein, 14 aa, Artificial
  synthetic peptide
  MOD_RES (1)..(1): tetramethylguanyl-arginine
  MOD_RES (3)..(3): 3-((2-naphthyl) alanine
  MOD_RES (6)..(6): citrulline
  MOD_RES (8)..(8): D-citrulline
  MOD_RES (12)..(12): citrulline
  MOD_RES (14)..(14): C′ AMIDATED
XRXCYXKXPYRXCR

SEQ ID NO: 47 - protein, 14 aa, Artificial
  synthetic peptide
  MOD_RES (1)..(1): 6-aminohexanoyl-arginine
  MOD_RES (3)..(3): 3-((2-naphthyl) alanine
  MOD_RES (6)..(6): citrulline
  MOD_RES (8)..(8): D-citrulline
  MOD_RES (12)..(12): citrulline
  MOD_RES (14)..(14): C′ AMIDATED
XRXCYXKXPYRXCR

SEQ ID NO: 48 - protein, 14 aa, Artificial
  synthetic peptide
  MOD_RES (1)..(1): 6-aminohexanoyl-arginine
  MOD_RES (3)..(3): 3-((2-naphthyl) alanine
  MOD_RES (8)..(8): D-lysine
  MOD_RES (12)..(12): citrulline
XRXCYRKXPYRXCR

SEQ ID NO: 49 - protein, 14 aa, Artificial
  synthetic peptide
  MOD_RES (3)..(3): 3-((2-naphthyl) alanine
  MOD_RES (6)..(6): citrulline
  MOD_RES (8)..(8): D-lysine
  MOD_RES (12)..(12): citrulline
  MOD_RES (14)..(14): C′ amidated
RRXCYXRXPYRXCR

SEQ ID NO: 50 - protein, 14 aa, Artificial
  synthetic peptide
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-lysine
  MOD_RES (12)..(12): citrulline
  MOD_RES (14)..(14): C′ amidated
RRXCYXRXPYRXCR

SEQ ID NO: 51 - protein, 14 aa, Artificial
  SYNTHETIC PEPTIDE
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-lysine
  MOD_RES (12)..(12): citrulline
  MOD_RES (14)..(14): C′ AMIDATED
RRXCYXKXPYRXCR

SEQ ID NO: 52 - protein, 14 aa, Artificial
  synthetic peptide
  MOD_RES (1)..(1): N′ ACETYLATED
  MOD_RES (3)..(3): 3-((2-naphthyl) alanine
  MOD_RES (8)..(8): D-citrulline
  MOD_RES (12)..(12): citrulline
  MOD_RES (14)..(14): C′ AMIDATED
RRXCYRKXPYRXCR

SEQ ID NO: 53 - protein, 14 aa, Artificial
  synthetic peptide
  MOD_RES (1)..(1): 4-fluorobenzoyl-arginine
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MISC_FEATURE (14)..(14): derivatization by a NH-methyl group
XRXCYXKXPYRXCR

SEQ ID NO: 54 - protein, 14 aa, Artificial
  synthetic peptide
  MOD_RES (1)..(1): 4-fluorobenzoyl-arginine
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MISC_FEATURE (14)..(14): derivatization by a NH-ethyl group
XRXCYXKXPYRXCR

SEQ ID NO: 55 - protein, 14 aa, Artificial
  synthetic peptide
  MOD_RES (1)..(1): 4-fluorobenzoyl-arginine
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MISC_FEATURE (14)..(14): derivatization by NH-isopropyl
XRXCYXKXPYRXCR

SEQ ID NO: 56 - protein, 14 aa, Artificial
  synthetic peptide
  MOD_RES (1)..(1): 4-fluorobenzoyl-arginine
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MISC_FEATURE (14)..(14): derivatization with a tyramine residue
XRXCYXKXPYRXCR

SEQ ID NO: 57 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
ARXCYRKXPYRXCR

SEQ ID NO: 58 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYAKXPYRXCR

SEQ ID NO: 59 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYRAXPYRXCR

SEQ ID NO: 60 - protein, 14 aa, Artificial
  synthetic peptide
  MOD_RES (3)..(3): 3-((2-naphthyl) alanine
  MOD_RES (8)..(8): D-alanine
  MOD_RES (12)..(12): citrulline
RRXCYRKXPYRXCR

SEQ ID NO: 61 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYRKXAYRXCR

SEQ ID NO: 62 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYRKXPARXCR

SEQ ID NO: 63 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYRKXPYAXCR

SEQ ID NO: 64 - protein, 14 aa, Artificial
  synthetic peptide
  MOD_RES (1)..(1): citrulline
  MOD_RES (3)..(3): 3-((2-naphthyl) alanine
  MOD_RES (8)..(8): D-lysine
  MOD_RES (12)..(12): citrulline
XRXCYRKXPYRXCR

SEQ ID NO: 65 - protein, 14 aa, Artificial
  synthetic peptide
  MOD_RES (3)..(3): 3-((2-naphthyl) alanine
  MOD_RES (6)..(6): citrulline
  MOD_RES (8)..(8): D-lysine
  MOD_RES (12)..(12): citrulline
RRXCYXKXPYRXCR

SEQ ID NO: 66 - protein, 14 aa, Artificial
  synthetic peptide
  MOD_RES (3)..(3): 3-((2-naphthyl) alanine
  MOD_RES (6)..(6): citrulline
  MOD_RES (8)..(8): D-lysine
  MOD_RES (12)..(12): citrulline
  MOD_RES (14)..(14): C′ AMIDATED
RRXCYXKXPYRXCR

SEQ ID NO: 67 - protein, 14 aa, Artificial
  synthetic peptide
  MOD_RES (3)..(3): 3-((2-naphthyl) alanine
  MOD_RES (7)..(7): citrulline
  MOD_RES (8)..(8): D-lysine
  MOD_RES (12)..(12): citrulline
RRXCYRXXPYRXCR

SEQ ID NO: 68 - protein, 14 aa, Artificial
  synthetic peptide
  MOD_RES (3)..(3): 3-((2-naphthyl) alanine
  MOD_RES (6)..(6): citrulline
  MOD_RES (8)..(8): D-citrulline
  MOD_RES (12)..(12): citrulline
  MOD_RES (14)..(14): C′ AMIDATED
RRXCYXKXPYRXCR

SEQ ID NO: 69 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYRKXPYRXCR

SEQ ID NO: 70 - protein, 14 aa, Artificial
  synthetic peptide
  MOD_RES (3)..(3): 3-((2-naphthyl) alanine
  MOD_RES (6)..(6): citrulline
  MOD_RES (8)..(8): D-citrulline
  MOD_RES (12)..(12): citrulline
RRXCYXKXPYRXCR

SEQ ID NO: 71 - protein, 14 aa, Artificial
  synthetic peptide
  MOD_RES (3)..(3): 3-((2-naphthyl) alanine
  MOD_RES (8)..(8): D-citrulline
  MOD_RES (12)..(12): citrulline
RRXCYRKXPYRXCR

SEQ ID NO: 72 - protein, 14 aa, Artificial
  synthetic peptide
  MOD_RES (1)..(1): citrulline
  MOD_RES (3)..(3): 3-((2-naphthyl) alanine
  MOD_RES (8)..(8): D-citrulline
  MOD_RES (12)..(12): citrulline
  MOD_RES (14)..(14): C′ Amidated
XRXCYRKXPYRXCR